SEQUENCE LISTING (26 sequences)

SEQ ID NO: 1 - protein, 370 aa, Saccharomyces cerevisiae
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPVGFKNGTDGTLNVAVDACQAAAHSHHFMGVTKHGVAAITTTKGNEHCFVILRGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 2 - protein, 370 aa, Saccharomyces cerevisiae
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPVGFKNGTDGTLNVAVDACQAAAHSHHFMGVTLHGVAAITTTKGNEHCFVILRGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 3 - protein, 370 aa, Saccharomyces cerevisiae
MFIKNDHAGDRKRLEDWRIKGYDPLTPPDLLQHEFPISAKGEENIIKARDSVCDILNGKDDRLVIVIGPCSLHDPKAAYDYADRLAKISEKLSKDLLIIMRAYLEKPRTTVGWKGLINDPDMNNSFQINKGLRISREMFIKLVEKLPIAGEMLDTISPQFLSDCFSLGAIGARTTESQLHRELASGLSFPIGFKNGTDGGLQVAIDAMRAAAHEHYFLSVTKPGVTAIVGTEGNKDTFLILRGGKNGTNFDKESVQNTKKQLEKAGLTDDSQKRIMIDCSHGNSNKDFKNQPKVAKCIYDQLTEGENSLCGVMIESNINEGRQDIPKEGGREGLKYGCSVTDACIGWESTEQVLELLAEGVRNRRKALKK

SEQ ID NO: 4 - protein, 256 aa, Saccharomyces cerevisiae
MDFTKPETVLNLQNIRDELVRMEDSIIFKFIERSHFATCPSVYEANHPGLEIPNFKGSFLDWALSNLEIAHSRIRRFESPDETPFFPDKIQKSFLPSINYPQILAPYAPEVNYNDKIKKVYIEKIIPLISKRDGDDKNNFGSVATRDIECLQSLSRRIHFGKFVAEAKFQSDIPLYTKLIKSKDVEGIMKNITNSAVEEKILERLTKKAEVYGVDPTNESGERRITPEYLVKIYKEIVIPITKEVEVEYLLRRLEE

SEQ ID NO: 5 - protein, 256 aa, Saccharomyces cerevisiae
MDFTKPETVLNLQNIRDELVRMEDSIIFKFIERSHFATCPSVYEANHPGLEIPNFKGSFLDWALSNLEIAHSRIRRFESPDETPFFPDKIQKSFLPSINYPQILAPYAPEVNYNDKIKKVYIEKIIPLISKRDGDDKNNFGSVATRDIECLQSLSRRIHFGKFVAEAKFQSDIPLYTKLIKSKDVEGIMKNITNSAVEEKILERLTKKAEVYGVDPTNESGERRIIPEYLVKIYKEIVIPITKEVEVEYLLRRLEE

SEQ ID NO: 6 - protein, 2233 aa, Saccharomyces cerevisiae
MSEESLFESSPQKMEYEITNYSERHTELPGHFIGLNTVDKLEESPLRDFVKSHGGHTVISKILIANNGIAAVKEIRSVRKWAYETFGDDRTVQFVAMATPEDLEANAEYIRMADQYIEVPGGTNNNNYANVDLIVDIAERADVDAVWAGWGHASENPLLPEKLSQSKRKVIFIGPPGNAMRSLGDKISSTIVAQSAKVPCIPWSGTGVDTVHVDEKTGLVSVDDDIYQKGCCTSPEDGLQKAKRIGFPVMIKASEGGGGKGIRQVEREEDFIALYHQAANEIPGSPIFIMKLAGRARHLEVQLLADQYGTNISLFGRDCSVQRRHQKIIEEAPVTIAKAETFHEMEKAAVRLGKLVGYVSAGTVEYLYSHDDGKFYFLELNPRLQVEHPTTEMVSGVNLPAAQLQIAMGIPMHRISDIRTLYGMNPHSASEIDFEFKTQDATKKQRRPIPKGHCTACRITSEDPNDGFKPSGGTLHELNFRSSSNVWGYFSVGNNGNIHSFSDSQFGHIFAFGENRQASRKHMVVALKELSIRGDFRTTVEYLIKLLETEDFEDNTITTGWLDDLITHKMTAEKPDPTLAVICGAATKAFLASEEARHKYIESLQKGQVLSKDLLQTMFPVDFIHEGKRYKFTVAKSGNDRYTLFINGSKCDIILRQLSDGGLLIAIGGKSHTIYWKEEVAATRLSVDSMTTLLEVENDPTQLRTPSPGKLVKFLVENGEHIIKGQPYAEIEVMKMQMPLVSQENGIVQLLKQPGSTIVAGDIMAIMTLDDPSKVKHALPFEGMLPDFGSPVIEGTKPAYKFKSLVSTLENILKGYDNQVIMNASLQQLIEVLRNPKLPYSEWKLHISALHSRLPAKLDEQMEELVARSLRRGAVFPARQLSKLIDMAVKNPEYNPDKLLGAVVEPLADIAHKYSNGLEAHEHSIFVHFLEEYYEVEKLFNGPNVREENIILKLRDENPKDLDKVALTVLSHSKVSAKNNLILAILKHYQPLCKLSSKVSAIFSTPLQHIVELESKATAKVALQAREILIQGALPSVKERTEQIEHILKSSVVKVAYGSSNPKRSEPDLNILKDLIDSNYVVFDVLLQFLTHQDPVVTAAAAQVYIRRAYRAYTIGDIRVHEGVTVPIVEWKFQLPSAAFSTFPTVKSKMGMNRAVSVSDLSYVANSQSSPLREGILMAVDHLDDVDEILSQSLEVIPRHQSSSNGPAPDRSGSSASLSNVANVCVASTEGFESEEEILVRLREILDLNKQELINASIRRITFMFGFKDGSYPKYYTFNGPNYNENETIRHIEPALAFQLELGRLSNFNIKPIFTDNRNIHVYEAVSKTSPLDKRFFTRGIIRTGHIRDDISIQEYLTSEANRLMSDILDNLEVTDTSNSDLNHIFINFIAVFDISPEDVEAAFGGFLERFGKRLLRLRVSSAEIRIIIKDPQTGAPVPLRALINNVSGYVIKTEMYTEVKNAKGEWVFKSLGKPGSMHLRPIATPYPVKEWLQPKRYKAHLMGTTYVYDFPELFRQASSSQWKNFSADVKLTDDFFISNELIEDENGELTEVEREPGANAIGMVAFKITVKTPEYPRGRQFVVVANDITFKIGSFGPQEDEFFNKVTEYARKRGIPRIYLAANSGARIGMAEEIVPLFQVAWNDAANPDKGFQYLYLTSEGMETLKKFDKENSVLTERTVINGEERFVIKTIIGSEDGLGVECLRGSGLIAGATSRAYHDIFTITLVTCRSVGIGAYLVRLGQRAIQVEGQPIILTGAPAINKMLGREVYTSNLQLGGTQIMYNNGVSHLTAVDDLAGVEKIVEWMSYVPAKRNMPVPILETKDTWDRPVDFTPTNDETYDVRWMIEGRETESGFEYGLFDKGSFFETLSGWAKGVVVGRARLGGIPLGVIGVETRTVENLIPADPANPNSAETLIQEPGQVWHPNSAFKTAQAINDFNNGEQLPMMILANWRGFSGGQRDMFNEVLKYGSFIVDALVDYKQPIIIYIPPTGELRGGSWVVVDPTINADQMEMYADVNARAGVLEPQGMVGIKFRREKLLDTMNRLDDKYRELRSQLSNKSLAPEVHQQISKQLADRERELLPIYGQISLQFADLHDRSSRMVAKGVISKELEWTEARRFFFWRLRRRLNEEYLIKRLSHQVGEASRLEKIARIRSWYPASVDHEDDRQVATWIEENYKTLDDKLKGLKLESFAQDLAKKIRSDHDNAIDGLSEVIKMLSTDDKEKLLKTLK

SEQ ID NO: 7 - protein, 2233 aa, Artificial Sequence
  S. cerevisiae ACC1 S659A
MSEESLFESSPQKMEYEITNYSERHTELPGHFIGLNTVDKLEESPLRDFVKSHGGHTVISKILIANNGIAAVKEIRSVRKWAYETFGDDRTVQFVAMATPEDLEANAEYIRMADQYIEVPGGTNNNNYANVDLIVDIAERADVDAVWAGWGHASENPLLPEKLSQSKRKVIFIGPPGNAMRSLGDKISSTIVAQSAKVPCIPWSGTGVDTVHVDEKTGLVSVDDDIYQKGCCTSPEDGLQKAKRIGFPVMIKASEGGGGKGIRQVEREEDFIALYHQAANEIPGSPIFIMKLAGRARHLEVQLLADQYGTNISLFGRDCSVQRRHQKIIEEAPVTIAKAETFHEMEKAAVRLGKLVGYVSAGTVEYLYSHDDGKFYFLELNPRLQVEHPTTEMVSGVNLPAAQLQIAMGIPMHRISDIRTLYGMNPHSASEIDFEFKTQDATKKQRRPIPKGHCTACRITSEDPNDGFKPSGGTLHELNFRSSSNVWGYFSVGNNGNIHSFSDSQFGHIFAFGENRQASRKHMVVALKELSIRGDFRTTVEYLIKLLETEDFEDNTITTGWLDDLITHKMTAEKPDPTLAVICGAATKAFLASEEARHKYIESLQKGQVLSKDLLQTMFPVDFIHEGKRYKFTVAKSGNDRYTLFINGSKCDIILRQLADGGLLIAIGGKSHTIYWKEEVAATRLSVDSMTTLLEVENDPTQLRTPSPGKLVKFLVENGEHIIKGQPYAEIEVMKMQMPLVSQENGIVQLLKQPGSTIVAGDIMAIMTLDDPSKVKHALPFEGMLPDFGSPVIEGTKPAYKFKSLVSTLENILKGYDNQVIMNASLQQLIEVLRNPKLPYSEWKLHISALHSRLPAKLDEQMEELVARSLRRGAVFPARQLSKLIDMAVKNPEYNPDKLLGAVVEPLADIAHKYSNGLEAHEHSIFVHFLEEYYEVEKLFNGPNVREENIILKLRDENPKDLDKVALTVLSHSKVSAKNNLILAILKHYQPLCKLSSKVSAIFSTPLQHIVELESKATAKVALQAREILIQGALPSVKERTEQIEHILKSSVVKVAYGSSNPKRSEPDLNILKDLIDSNYVVFDVLLQFLTHQDPVVTAAAAQVYIRRAYRAYTIGDIRVHEGVTVPIVEWKFQLPSAAFSTFPTVKSKMGMNRAVSVSDLSYVANSQSSPLREGILMAVDHLDDVDEILSQSLEVIPRHQSSSNGPAPDRSGSSASLSNVANVCVASTEGFESEEEILVRLREILDLNKQELINASIRRITFMFGFKDGSYPKYYTFNGPNYNENETIRHIEPALAFQLELGRLSNFNIKPIFTDNRNIHVYEAVSKTSPLDKRFFTRGIIRTGHIRDDISIQEYLTSEANRLMSDILDNLEVTDTSNSDLNHIFINFIAVFDISPEDVEAAFGGFLERFGKRLLRLRVSSAEIRIIIKDPQTGAPVPLRALINNVSGYVIKTEMYTEVKNAKGEWVFKSLGKPGSMHLRPIATPYPVKEWLQPKRYKAHLMGTTYVYDFPELFRQASSSQWKNFSADVKLTDDFFISNELIEDENGELTEVEREPGANAIGMVAFKITVKTPEYPRGRQFVVVANDITFKIGSFGPQEDEFFNKVTEYARKRGIPRIYLAANSGARIGMAEEIVPLFQVAWNDAANPDKGFQYLYLTSEGMETLKKFDKENSVLTERTVINGEERFVIKTIIGSEDGLGVECLRGSGLIAGATSRAYHDIFTITLVTCRSVGIGAYLVRLGQRAIQVEGQPIILTGAPAINKMLGREVYTSNLQLGGTQIMYNNGVSHLTAVDDLAGVEKIVEWMSYVPAKRNMPVPILETKDTWDRPVDFTPTNDETYDVRWMIEGRETESGFEYGLFDKGSFFETLSGWAKGVVVGRARLGGIPLGVIGVETRTVENLIPADPANPNSAETLIQEPGQVWHPNSAFKTAQAINDFNNGEQLPMMILANWRGFSGGQRDMFNEVLKYGSFIVDALVDYKQPIIIYIPPTGELRGGSWVVVDPTINADQMEMYADVNARAGVLEPQGMVGIKFRREKLLDTMNRLDDKYRELRSQLSNKSLAPEVHQQISKQLADRERELLPIYGQISLQFADLHDRSSRMVAKGVISKELEWTEARRFFFWRLRRRLNEEYLIKRLSHQVGEASRLEKIARIRSWYPASVDHEDDRQVATWIEENYKTLDDKLKGLKLESFAQDLAKKIRSDHDNAIDGLSEVIKMLSTDDKEKLLKTLK

SEQ ID NO: 8 - protein, 2233 aa, Saccharomyces cerevisiae
MSEESLFESSPQKMEYEITNYSERHTELPGHFIGLNTVDKLEESPLRDFVKSHGGHTVISKILIANNGIAAVKEIRSVRKWAYETFGDDRTVQFVAMATPEDLEANAEYIRMADQYIEVPGGTNNNNYANVDLIVDIAERADVDAVWAGWGHASENPLLPEKLSQSKRKVIFIGPPGNAMRSLGDKISSTIVAQSAKVPCIPWSGTGVDTVHVDEKTGLVSVDDDIYQKGCCTSPEDGLQKAKRIGFPVMIKASEGGGGKGIRQVEREEDFIALYHQAANEIPGSPIFIMKLAGRARHLEVQLLADQYGTNISLFGRDCSVQRRHQKIIEEAPVTIAKAETFHEMEKAAVRLGKLVGYVSAGTVEYLYSHDDGKFYFLELNPRLQVEHPTTEMVSGVNLPAAQLQIAMGIPMHRISDIRTLYGMNPHSASEIDFEFKTQDATKKQRRPIPKGHCTACRITSEDPNDGFKPSGGTLHELNFRSSSNVWGYFSVGNNGNIHSFSDSQFGHIFAFGENRQASRKHMVVALKELSIRGDFRTTVEYLIKLLETEDFEDNTITTGWLDDLITHKMTAEKPDPTLAVICGAATKAFLASEEARHKYIESLQKGQVLSKDLLQTMFPVDFIHEGKRYKFTVAKSGNDRYTLFINGSKCDIILRQLVDGGLLIAIGGKSHTIYWKEEVAATRLSVDSMTTLLEVENDPTQLRTPSPGKLVKFLVENGEHIIKGQPYAEIEVMKMQMPLVSQENGIVQLLKQPGSTIVAGDIMAIMTLDDPSKVKHALPFEGMLPDFGSPVIEGTKPAYKFKSLVSTLENILKGYDNQVIMNASLQQLIEVLRNPKLPYSEWKLHISALHSRLPAKLDEQMEELVARSLRRGAVFPARQLSKLIDMAVKNPEYNPDKLLGAVVEPLADIAHKYSNGLEAHEHSIFVHFLEEYYEVEKLFNGPNVREENIILKLRDENPKDLDKVALTVLSHSKVSAKNNLILAILKHYQPLCKLSSKVSAIFSTPLQHIVELESKATAKVALQAREILIQGALPSVKERTEQIEHILKSSVVKVAYGSSNPKRSEPDLNILKDLIDSNYVVFDVLLQFLTHQDPVVTAAAAQVYIRRAYRAYTIGDIRVHEGVTVPIVEWKFQLPSAAFSTFPTVKSKMGMNRAVSVSDLSYVANSQSSPLREGILMAVDHLDDVDEILSQSLEVIPRHQSSSNGPAPDRSGSSASLSNVANVCVASTEGFESEEEILVRLREILDLNKQELINASIRRITFMFGFKDGSYPKYYTFNGPNYNENETIRHIEPALAFQLELGRLSNFNIKPIFTDNRNIHVYEAVSKTSPLDKRFFTRGIIRTGHIRDDISIQEYLTSEANRLMSDILDNLEVTDTSNSDLNHIFINFIAVFDISPEDVEAAFGGFLERFGKRLLRLRVSSAEIRIIIKDPQTGAPVPLRALINNVSGYVIKTEMYTEVKNAKGEWVFKSLGKPGSMHLRPIATPYPVKEWLQPKRYKAHLMGTTYVYDFPELFRQASSSQWKNFSADVKLTDDFFISNELIEDENGELTEVEREPGANAIGMVAFKITVKTPEYPRGRQFVVVANDITFKIGSFGPQEDEFFNKVTEYARKRGIPRIYLAANSGARIGMAEEIVPLFQVAWNDAANPDKGFQYLYLTSEGMETLKKFDKENSVLTERTVINGEERFVIKTIIGSEDGLGVECLRGSGLIAGATSRAYHDIFTITLVTCRSVGIGAYLVRLGQRAIQVEGQPIILTGAPAINKMLGREVYTSNLQLGGTQIMYNNGVSHLTAVDDLAGVEKIVEWMSYVPAKRNMPVPILETKDTWDRPVDFTPTNDETYDVRWMIEGRETESGFEYGLFDKGSFFETLSGWAKGVVVGRARLGGIPLGVIGVETRTVENLIPADPANPNSAETLIQEPGQVWHPNSAFKTAQAINDFNNGEQLPMMILANWRGFSGGQRDMFNEVLKYGSFIVDALVDYKQPIIIYIPPTGELRGGSWVVVDPTINADQMEMYADVNARAGVLEPQGMVGIKFRREKLLDTMNRLDDKYRELRSQLSNKSLAPEVHQQISKQLADRERELLPIYGQISLQFADLHDRSSRMVAKGVISKELEWTEARRFFFWRLRRRLNEEYLIKRLSHQVGEASRLEKIARIRSWYPASVDHEDDRQVATWIEENYKTLDDKLKGLKLESFAQDLAKKIRSDHDNAIDGLSEVIKMLSTDDKEKLLKTLK

SEQ ID NO: 9 - protein, 2233 aa, Saccharomyces cerevisiae
MSEESLFESSPQKMEYEITNYSERHTELPGHFIGLNTVDKLEESPLRDFVKSHGGHTVISKILIANNGIAAVKEIRSVRKWAYETFGDDRTVQFVAMATPEDLEANAEYIRMADQYIEVPGGTNNNNYANVDLIVDIAERADVDAVWAGWGHASENPLLPEKLSQSKRKVIFIGPPGNAMRSLGDKISSTIVAQSAKVPCIPWSGTGVDTVHVDEKTGLVSVDDDIYQKGCCTSPEDGLQKAKRIGFPVMIKASEGGGGKGIRQVEREEDFIALYHQAANEIPGSPIFIMKLAGRARHLEVQLLADQYGTNISLFGRDCSVQRRHQKIIEEAPVTIAKAETFHEMEKAAVRLGKLVGYVSAGTVEYLYSHDDGKFYFLELNPRLQVEHPTTEMVSGVNLPAAQLQIAMGIPMHRISDIRTLYGMNPHSASEIDFEFKTQDATKKQRRPIPKGHCTACRITSEDPNDGFKPSGGTLHELNFRSSSNVWGYFSVGNNGNIHSFSDSQFGHIFAFGENRQASRKHMVVALKELSIRGDFRTTVEYLIKLLETEDFEDNTITTGWLDDLITHKMTAEKPDPTLAVICGAATKAFLASEEARHKYIESLQKGQVLSKDLLQTMFPVDFIHEGKRYKFTVAKSGNDRYTLFINGSKCDIILRQLSDGGLLIAIGGKSHTIYWKEEVAATRLSVDSMTTLLEVENDPTQLRTPSPGKLVKFLVENGEHIIKGQPYAEIEVMKMQMPLVSQENGIVQLLKQPGSTIVAGDIMAIMTLDDPSKVKHALPFEGMLPDFGSPVIEGTKPAYKFKSLVSTLENILKGYDNQVIMNASLQQLIEVLRNPKLPYSEWKLHISALHSRLPAKLDEQMEELVARSLRRGAVFPARQLSKLIDMAVKNPEYNPDKLLGAVVEPLADIAHKYSNGLEAHEHSIFVHFLEEYYEVEKLFNGPNVREENIILKLRDENPKDLDKVALTVLSHSKVSAKNNLILAILKHYQPLCKLSSKVSAIFSTPLQHIVELESKATAKVALQAREILIQGALPSVKERTEQIEHILKSSVVKVAYGSSNPKRSEPDLNILKDLIDSNYVVFDVLLQFLTHQDPVVTAAAAQVYIRRAYRAYTIGDIRVHEGVTVPIVEWKFQLPSAAFSTFPTVKSKMGMNRAVAVSDLSYVANSQSSPLREGILMAVDHLDDVDEILSQSLEVIPRHQSSSNGPAPDRSGSSASLSNVANVCVASTEGFESEEEILVRLREILDLNKQELINASIRRITFMFGFKDGSYPKYYTFNGPNYNENETIRHIEPALAFQLELGRLSNFNIKPIFTDNRNIHVYEAVSKTSPLDKRFFTRGIIRTGHIRDDISIQEYLTSEANRLMSDILDNLEVTDTSNSDLNHIFINFIAVFDISPEDVEAAFGGFLERFGKRLLRLRVSSAEIRIIIKDPQTGAPVPLRALINNVSGYVIKTEMYTEVKNAKGEWVFKSLGKPGSMHLRPIATPYPVKEWLQPKRYKAHLMGTTYVYDFPELFRQASSSQWKNFSADVKLTDDFFISNELIEDENGELTEVEREPGANAIGMVAFKITVKTPEYPRGRQFVVVANDITFKIGSFGPQEDEFFNKVTEYARKRGIPRIYLAANSGARIGMAEEIVPLFQVAWNDAANPDKGFQYLYLTSEGMETLKKFDKENSVLTERTVINGEERFVIKTIIGSEDGLGVECLRGSGLIAGATSRAYHDIFTITLVTCRSVGIGAYLVRLGQRAIQVEGQPIILTGAPAINKMLGREVYTSNLQLGGTQIMYNNGVSHLTAVDDLAGVEKIVEWMSYVPAKRNMPVPILETKDTWDRPVDFTPTNDETYDVRWMIEGRETESGFEYGLFDKGSFFETLSGWAKGVVVGRARLGGIPLGVIGVETRTVENLIPADPANPNSAETLIQEPGQVWHPNSAFKTAQAINDFNNGEQLPMMILANWRGFSGGQRDMFNEVLKYGSFIVDALVDYKQPIIIYIPPTGELRGGSWVVVDPTINADQMEMYADVNARAGVLEPQGMVGIKFRREKLLDTMNRLDDKYRELRSQLSNKSLAPEVHQQISKQLADRERELLPIYGQISLQFADLHDRSSRMVAKGVISKELEWTEARRFFFWRLRRRLNEEYLIKRLSHQVGEASRLEKIARIRSWYPASVDHEDDRQVATWIEENYKTLDDKLKGLKLESFAQDLAKKIRSDHDNAIDGLSEVIKMLSTDDKEKLLKTLK

SEQ ID NO: 10 - protein, 2233 aa, Saccharomyces cerevisiae
MSEESLFESSPQKMEYEITNYSERHTELPGHFIGLNTVDKLEESPLRDFVKSHGGHTVISKILIANNGIAAVKEIRSVRKWAYETFGDDRTVQFVAMATPEDLEANAEYIRMADQYIEVPGGTNNNNYANVDLIVDIAERADVDAVWAGWGHASENPLLPEKLSQSKRKVIFIGPPGNAMRSLGDKISSTIVAQSAKVPCIPWSGTGVDTVHVDEKTGLVSVDDDIYQKGCCTSPEDGLQKAKRIGFPVMIKASEGGGGKGIRQVEREEDFIALYHQAANEIPGSPIFIMKLAGRARHLEVQLLADQYGTNISLFGRDCSVQRRHQKIIEEAPVTIAKAETFHEMEKAAVRLGKLVGYVSAGTVEYLYSHDDGKFYFLELNPRLQVEHPTTEMVSGVNLPAAQLQIAMGIPMHRISDIRTLYGMNPHSASEIDFEFKTQDATKKQRRPIPKGHCTACRITSEDPNDGFKPSGGTLHELNFRSSSNVWGYFSVGNNGNIHSFSDSQFGHIFAFGENRQASRKHMVVALKELSIRGDFRTTVEYLIKLLETEDFEDNTITTGWLDDLITHKMTAEKPDPTLAVICGAATKAFLASEEARHKYIESLQKGQVLSKDLLQTMFPVDFIHEGKRYKFTVAKSGNDRYTLFINGSKCDIILRQLSDGGLLIAIGGKSHTIYWKEEVAATRLSVDSMTTLLEVENDPTQLRTPSPGKLVKFLVENGEHIIKGQPYAEIEVMKMQMPLVSQENGIVQLLKQPGSTIVAGDIMAIMTLDDPSKVKHALPFEGMLPDFGSPVIEGTKPAYKFKSLVSTLENILKGYDNQVIMNASLQQLIEVLRNPKLPYSEWKLHISALHSRLPAKLDEQMEELVARSLRRGAVFPARQLSKLIDMAVKNPEYNPDKLLGAVVEPLADIAHKYSNGLEAHEHSIFVHFLEEYYEVEKLFNGPNVREENIILKLRDENPKDLDKVALTVLSHSKVSAKNNLILAILKHYQPLCKLSSKVSAIFSTPLQHIVELESKATAKVALQAREILIQGALPSVKERTEQIEHILKSSVVKVAYGSSNPKRSEPDLNILKDLIDSNYVVFDVLLQFLTHQDPVVTAAAAQVYIRRAYRAYTIGDIRVHEGVTVPIVEWKFQLPSAAFSTFPTVKSKMGMNRAVVVSDLSYVANSQSSPLREGILMAVDHLDDVDEILSQSLEVIPRHQSSSNGPAPDRSGSSASLSNVANVCVASTEGFESEEEILVRLREILDLNKQELINASIRRITFMFGFKDGSYPKYYTFNGPNYNENETIRHIEPALAFQLELGRLSNFNIKPIFTDNRNIHVYEAVSKTSPLDKRFFTRGIIRTGHIRDDISIQEYLTSEANRLMSDILDNLEVTDTSNSDLNHIFINFIAVFDISPEDVEAAFGGFLERFGKRLLRLRVSSAEIRIIIKDPQTGAPVPLRALINNVSGYVIKTEMYTEVKNAKGEWVFKSLGKPGSMHLRPIATPYPVKEWLQPKRYKAHLMGTTYVYDFPELFRQASSSQWKNFSADVKLTDDFFISNELIEDENGELTEVEREPGANAIGMVAFKITVKTPEYPRGRQFVVVANDITFKIGSFGPQEDEFFNKVTEYARKRGIPRIYLAANSGARIGMAEEIVPLFQVAWNDAANPDKGFQYLYLTSEGMETLKKFDKENSVLTERTVINGEERFVIKTIIGSEDGLGVECLRGSGLIAGATSRAYHDIFTITLVTCRSVGIGAYLVRLGQRAIQVEGQPIILTGAPAINKMLGREVYTSNLQLGGTQIMYNNGVSHLTAVDDLAGVEKIVEWMSYVPAKRNMPVPILETKDTWDRPVDFTPTNDETYDVRWMIEGRETESGFEYGLFDKGSFFETLSGWAKGVVVGRARLGGIPLGVIGVETRTVENLIPADPANPNSAETLIQEPGQVWHPNSAFKTAQAINDFNNGEQLPMMILANWRGFSGGQRDMFNEVLKYGSFIVDALVDYKQPIIIYIPPTGELRGGSWVVVDPTINADQMEMYADVNARAGVLEPQGMVGIKFRREKLLDTMNRLDDKYRELRSQLSNKSLAPEVHQQISKQLADRERELLPIYGQISLQFADLHDRSSRMVAKGVISKELEWTEARRFFFWRLRRRLNEEYLIKRLSHQVGEASRLEKIARIRSWYPASVDHEDDRQVATWIEENYKTLDDKLKGLKLESFAQDLAKKIRSDHDNAIDGLSEVIKMLSTDDKEKLLKTLK

SEQ ID NO: 11 - protein, 2233 aa, Saccharomyces cerevisiae
MSEESLFESSPQKMEYEITNYSERHTELPGHFIGLNTVDKLEESPLRDFVKSHGGHTVISKILIANNGIAAVKEIRSVRKWAYETFGDDRTVQFVAMATPEDLEANAEYIRMADQYIEVPGGTNNNNYANVDLIVDIAERADVDAVWAGWGHASENPLLPEKLSQSKRKVIFIGPPGNAMRSLGDKISSTIVAQSAKVPCIPWSGTGVDTVHVDEKTGLVSVDDDIYQKGCCTSPEDGLQKAKRIGFPVMIKASEGGGGKGIRQVEREEDFIALYHQAANEIPGSPIFIMKLAGRARHLEVQLLADQYGTNISLFGRDCSVQRRHQKIIEEAPVTIAKAETFHEMEKAAVRLGKLVGYVSAGTVEYLYSHDDGKFYFLELNPRLQVEHPTTEMVSGVNLPAAQLQIAMGIPMHRISDIRTLYGMNPHSASEIDFEFKTQDATKKQRRPIPKGHCTACRITSEDPNDGFKPSGGTLHELNFRSSSNVWGYFSVGNNGNIHSFSDSQFGHIFAFGENRQASRKHMVVALKELSIRGDFRTTVEYLIKLLETEDFEDNTITTGWLDDLITHKMTAEKPDPTLAVICGAATKAFLASEEARHKYIESLQKGQVLSKDLLQTMFPVDFIHEGKRYKFTVAKSGNDRYTLFINGSKCDIILRQLADGGLLIAIGGKSHTIYWKEEVAATRLSVDSMTTLLEVENDPTQLRTPSPGKLVKFLVENGEHIIKGQPYAEIEVMKMQMPLVSQENGIVQLLKQPGSTIVAGDIMAIMTLDDPSKVKHALPFEGMLPDFGSPVIEGTKPAYKFKSLVSTLENILKGYDNQVIMNASLQQLIEVLRNPKLPYSEWKLHISALHSRLPAKLDEQMEELVARSLRRGAVFPARQLSKLIDMAVKNPEYNPDKLLGAVVEPLADIAHKYSNGLEAHEHSIFVHFLEEYYEVEKLFNGPNVREENIILKLRDENPKDLDKVALTVLSHSKVSAKNNLILAILKHYQPLCKLSSKVSAIFSTPLQHIVELESKATAKVALQAREILIQGALPSVKERTEQIEHILKSSVVKVAYGSSNPKRSEPDLNILKDLIDSNYVVFDVLLQFLTHQDPVVTAAAAQVYIRRAYRAYTIGDIRVHEGVTVPIVEWKFQLPSAAFSTFPTVKSKMGMNRAVAVSDLSYVANSQSSPLREGILMAVDHLDDVDEILSQSLEVIPRHQSSSNGPAPDRSGSSASLSNVANVCVASTEGFESEEEILVRLREILDLNKQELINASIRRITFMFGFKDGSYPKYYTFNGPNYNENETIRHIEPALAFQLELGRLSNFNIKPIFTDNRNIHVYEAVSKTSPLDKRFFTRGIIRTGHIRDDISIQEYLTSEANRLMSDILDNLEVTDTSNSDLNHIFINFIAVFDISPEDVEAAFGGFLERFGKRLLRLRVSSAEIRIIIKDPQTGAPVPLRALINNVSGYVIKTEMYTEVKNAKGEWVFKSLGKPGSMHLRPIATPYPVKEWLQPKRYKAHLMGTTYVYDFPELFRQASSSQWKNFSADVKLTDDFFISNELIEDENGELTEVEREPGANAIGMVAFKITVKTPEYPRGRQFVVVANDITFKIGSFGPQEDEFFNKVTEYARKRGIPRIYLAANSGARIGMAEEIVPLFQVAWNDAANPDKGFQYLYLTSEGMETLKKFDKENSVLTERTVINGEERFVIKTIIGSEDGLGVECLRGSGLIAGATSRAYHDIFTITLVTCRSVGIGAYLVRLGQRAIQVEGQPIILTGAPAINKMLGREVYTSNLQLGGTQIMYNNGVSHLTAVDDLAGVEKIVEWMSYVPAKRNMPVPILETKDTWDRPVDFTPTNDETYDVRWMIEGRETESGFEYGLFDKGSFFETLSGWAKGVVVGRARLGGIPLGVIGVETRTVENLIPADPANPNSAETLIQEPGQVWHPNSAFKTAQAINDFNNGEQLPMMILANWRGFSGGQRDMFNEVLKYGSFIVDALVDYKQPIIIYIPPTGELRGGSWVVVDPTINADQMEMYADVNARAGVLEPQGMVGIKFRREKLLDTMNRLDDKYRELRSQLSNKSLAPEVHQQISKQLADRERELLPIYGQISLQFADLHDRSSRMVAKGVISKELEWTEARRFFFWRLRRRLNEEYLIKRLSHQVGEASRLEKIARIRSWYPASVDHEDDRQVATWIEENYKTLDDKLKGLKLESFAQDLAKKIRSDHDNAIDGLSEVIKMLSTDDKEKLLKTLK

SEQ ID NO: 12 - protein, 2233 aa, Saccharomyces cerevisiae
MSEESLFESSPQKMEYEITNYSERHTELPGHFIGLNTVDKLEESPLRDFVKSHGGHTVISKILIANNGIAAVKEIRSVRKWAYETFGDDRTVQFVAMATPEDLEANAEYIRMADQYIEVPGGTNNNNYANVDLIVDIAERADVDAVWAGWGHASENPLLPEKLSQSKRKVIFIGPPGNAMRSLGDKISSTIVAQSAKVPCIPWSGTGVDTVHVDEKTGLVSVDDDIYQKGCCTSPEDGLQKAKRIGFPVMIKASEGGGGKGIRQVEREEDFIALYHQAANEIPGSPIFIMKLAGRARHLEVQLLADQYGTNISLFGRDCSVQRRHQKIIEEAPVTIAKAETFHEMEKAAVRLGKLVGYVSAGTVEYLYSHDDGKFYFLELNPRLQVEHPTTEMVSGVNLPAAQLQIAMGIPMHRISDIRTLYGMNPHSASEIDFEFKTQDATKKQRRPIPKGHCTACRITSEDPNDGFKPSGGTLHELNFRSSSNVWGYFSVGNNGNIHSFSDSQFGHIFAFGENRQASRKHMVVALKELSIRGDFRTTVEYLIKLLETEDFEDNTITTGWLDDLITHKMTAEKPDPTLAVICGAATKAFLASEEARHKYIESLQKGQVLSKDLLQTMFPVDFIHEGKRYKFTVAKSGNDRYTLFINGSKCDIILRQLADGGLLIAIGGKSHTIYWKEEVAATRLSVDSMTTLLEVENDPTQLRTPSPGKLVKFLVENGEHIIKGQPYAEIEVMKMQMPLVSQENGIVQLLKQPGSTIVAGDIMAIMTLDDPSKVKHALPFEGMLPDFGSPVIEGTKPAYKFKSLVSTLENILKGYDNQVIMNASLQQLIEVLRNPKLPYSEWKLHISALHSRLPAKLDEQMEELVARSLRRGAVFPARQLSKLIDMAVKNPEYNPDKLLGAVVEPLADIAHKYSNGLEAHEHSIFVHFLEEYYEVEKLFNGPNVREENIILKLRDENPKDLDKVALTVLSHSKVSAKNNLILAILKHYQPLCKLSSKVSAIFSTPLQHIVELESKATAKVALQAREILIQGALPSVKERTEQIEHILKSSVVKVAYGSSNPKRSEPDLNILKDLIDSNYVVFDVLLQFLTHQDPVVTAAAAQVYIRRAYRAYTIGDIRVHEGVTVPIVEWKFQLPSAAFSTFPTVKSKMGMNRAVVVSDLSYVANSQSSPLREGILMAVDHLDDVDEILSQSLEVIPRHQSSSNGPAPDRSGSSASLSNVANVCVASTEGFESEEEILVRLREILDLNKQELINASIRRITFMFGFKDGSYPKYYTFNGPNYNENETIRHIEPALAFQLELGRLSNFNIKPIFTDNRNIHVYEAVSKTSPLDKRFFTRGIIRTGHIRDDISIQEYLTSEANRLMSDILDNLEVTDTSNSDLNHIFINFIAVFDISPEDVEAAFGGFLERFGKRLLRLRVSSAEIRIIIKDPQTGAPVPLRALINNVSGYVIKTEMYTEVKNAKGEWVFKSLGKPGSMHLRPIATPYPVKEWLQPKRYKAHLMGTTYVYDFPELFRQASSSQWKNFSADVKLTDDFFISNELIEDENGELTEVEREPGANAIGMVAFKITVKTPEYPRGRQFVVVANDITFKIGSFGPQEDEFFNKVTEYARKRGIPRIYLAANSGARIGMAEEIVPLFQVAWNDAANPDKGFQYLYLTSEGMETLKKFDKENSVLTERTVINGEERFVIKTIIGSEDGLGVECLRGSGLIAGATSRAYHDIFTITLVTCRSVGIGAYLVRLGQRAIQVEGQPIILTGAPAINKMLGREVYTSNLQLGGTQIMYNNGVSHLTAVDDLAGVEKIVEWMSYVPAKRNMPVPILETKDTWDRPVDFTPTNDETYDVRWMIEGRETESGFEYGLFDKGSFFETLSGWAKGVVVGRARLGGIPLGVIGVETRTVENLIPADPANPNSAETLIQEPGQVWHPNSAFKTAQAINDFNNGEQLPMMILANWRGFSGGQRDMFNEVLKYGSFIVDALVDYKQPIIIYIPPTGELRGGSWVVVDPTINADQMEMYADVNARAGVLEPQGMVGIKFRREKLLDTMNRLDDKYRELRSQLSNKSLAPEVHQQISKQLADRERELLPIYGQISLQFADLHDRSSRMVAKGVISKELEWTEARRFFFWRLRRRLNEEYLIKRLSHQVGEASRLEKIARIRSWYPASVDHEDDRQVATWIEENYKTLDDKLKGLKLESFAQDLAKKIRSDHDNAIDGLSEVIKMLSTDDKEKLLKTLK

SEQ ID NO: 13 - protein, 2233 aa, Saccharomyces cerevisiae
MSEESLFESSPQKMEYEITNYSERHTELPGHFIGLNTVDKLEESPLRDFVKSHGGHTVISKILIANNGIAAVKEIRSVRKWAYETFGDDRTVQFVAMATPEDLEANAEYIRMADQYIEVPGGTNNNNYANVDLIVDIAERADVDAVWAGWGHASENPLLPEKLSQSKRKVIFIGPPGNAMRSLGDKISSTIVAQSAKVPCIPWSGTGVDTVHVDEKTGLVSVDDDIYQKGCCTSPEDGLQKAKRIGFPVMIKASEGGGGKGIRQVEREEDFIALYHQAANEIPGSPIFIMKLAGRARHLEVQLLADQYGTNISLFGRDCSVQRRHQKIIEEAPVTIAKAETFHEMEKAAVRLGKLVGYVSAGTVEYLYSHDDGKFYFLELNPRLQVEHPTTEMVSGVNLPAAQLQIAMGIPMHRISDIRTLYGMNPHSASEIDFEFKTQDATKKQRRPIPKGHCTACRITSEDPNDGFKPSGGTLHELNFRSSSNVWGYFSVGNNGNIHSFSDSQFGHIFAFGENRQASRKHMVVALKELSIRGDFRTTVEYLIKLLETEDFEDNTITTGWLDDLITHKMTAEKPDPTLAVICGAATKAFLASEEARHKYIESLQKGQVLSKDLLQTMFPVDFIHEGKRYKFTVAKSGNDRYTLFINGSKCDIILRQLVDGGLLIAIGGKSHTIYWKEEVAATRLSVDSMTTLLEVENDPTQLRTPSPGKLVKFLVENGEHIIKGQPYAEIEVMKMQMPLVSQENGIVQLLKQPGSTIVAGDIMAIMTLDDPSKVKHALPFEGMLPDFGSPVIEGTKPAYKFKSLVSTLENILKGYDNQVIMNASLQQLIEVLRNPKLPYSEWKLHISALHSRLPAKLDEQMEELVARSLRRGAVFPARQLSKLIDMAVKNPEYNPDKLLGAVVEPLADIAHKYSNGLEAHEHSIFVHFLEEYYEVEKLFNGPNVREENIILKLRDENPKDLDKVALTVLSHSKVSAKNNLILAILKHYQPLCKLSSKVSAIFSTPLQHIVELESKATAKVALQAREILIQGALPSVKERTEQIEHILKSSVVKVAYGSSNPKRSEPDLNILKDLIDSNYVVFDVLLQFLTHQDPVVTAAAAQVYIRRAYRAYTIGDIRVHEGVTVPIVEWKFQLPSAAFSTFPTVKSKMGMNRAVAVSDLSYVANSQSSPLREGILMAVDHLDDVDEILSQSLEVIPRHQSSSNGPAPDRSGSSASLSNVANVCVASTEGFESEEEILVRLREILDLNKQELINASIRRITFMFGFKDGSYPKYYTFNGPNYNENETIRHIEPALAFQLELGRLSNFNIKPIFTDNRNIHVYEAVSKTSPLDKRFFTRGIIRTGHIRDDISIQEYLTSEANRLMSDILDNLEVTDTSNSDLNHIFINFIAVFDISPEDVEAAFGGFLERFGKRLLRLRVSSAEIRIIIKDPQTGAPVPLRALINNVSGYVIKTEMYTEVKNAKGEWVFKSLGKPGSMHLRPIATPYPVKEWLQPKRYKAHLMGTTYVYDFPELFRQASSSQWKNFSADVKLTDDFFISNELIEDENGELTEVEREPGANAIGMVAFKITVKTPEYPRGRQFVVVANDITFKIGSFGPQEDEFFNKVTEYARKRGIPRIYLAANSGARIGMAEEIVPLFQVAWNDAANPDKGFQYLYLTSEGMETLKKFDKENSVLTERTVINGEERFVIKTIIGSEDGLGVECLRGSGLIAGATSRAYHDIFTITLVTCRSVGIGAYLVRLGQRAIQVEGQPIILTGAPAINKMLGREVYTSNLQLGGTQIMYNNGVSHLTAVDDLAGVEKIVEWMSYVPAKRNMPVPILETKDTWDRPVDFTPTNDETYDVRWMIEGRETESGFEYGLFDKGSFFETLSGWAKGVVVGRARLGGIPLGVIGVETRTVENLIPADPANPNSAETLIQEPGQVWHPNSAFKTAQAINDFNNGEQLPMMILANWRGFSGGQRDMFNEVLKYGSFIVDALVDYKQPIIIYIPPTGELRGGSWVVVDPTINADQMEMYADVNARAGVLEPQGMVGIKFRREKLLDTMNRLDDKYRELRSQLSNKSLAPEVHQQISKQLADRERELLPIYGQISLQFADLHDRSSRMVAKGVISKELEWTEARRFFFWRLRRRLNEEYLIKRLSHQVGEASRLEKIARIRSWYPASVDHEDDRQVATWIEENYKTLDDKLKGLKLESFAQDLAKKIRSDHDNAIDGLSEVIKMLSTDDKEKLLKTLK

SEQ ID NO: 14 - protein, 2233 aa, Saccharomyces cerevisiae
MSEESLFESSPQKMEYEITNYSERHTELPGHFIGLNTVDKLEESPLRDFVKSHGGHTVISKILIANNGIAAVKEIRSVRKWAYETFGDDRTVQFVAMATPEDLEANAEYIRMADQYIEVPGGTNNNNYANVDLIVDIAERADVDAVWAGWGHASENPLLPEKLSQSKRKVIFIGPPGNAMRSLGDKISSTIVAQSAKVPCIPWSGTGVDTVHVDEKTGLVSVDDDIYQKGCCTSPEDGLQKAKRIGFPVMIKASEGGGGKGIRQVEREEDFIALYHQAANEIPGSPIFIMKLAGRARHLEVQLLADQYGTNISLFGRDCSVQRRHQKIIEEAPVTIAKAETFHEMEKAAVRLGKLVGYVSAGTVEYLYSHDDGKFYFLELNPRLQVEHPTTEMVSGVNLPAAQLQIAMGIPMHRISDIRTLYGMNPHSASEIDFEFKTQDATKKQRRPIPKGHCTACRITSEDPNDGFKPSGGTLHELNFRSSSNVWGYFSVGNNGNIHSFSDSQFGHIFAFGENRQASRKHMVVALKELSIRGDFRTTVEYLIKLLETEDFEDNTITTGWLDDLITHKMTAEKPDPTLAVICGAATKAFLASEEARHKYIESLQKGQVLSKDLLQTMFPVDFIHEGKRYKFTVAKSGNDRYTLFINGSKCDIILRQLVDGGLLIAIGGKSHTIYWKEEVAATRLSVDSMTTLLEVENDPTQLRTPSPGKLVKFLVENGEHIIKGQPYAEIEVMKMQMPLVSQENGIVQLLKQPGSTIVAGDIMAIMTLDDPSKVKHALPFEGMLPDFGSPVIEGTKPAYKFKSLVSTLENILKGYDNQVIMNASLQQLIEVLRNPKLPYSEWKLHISALHSRLPAKLDEQMEELVARSLRRGAVFPARQLSKLIDMAVKNPEYNPDKLLGAVVEPLADIAHKYSNGLEAHEHSIFVHFLEEYYEVEKLFNGPNVREENIILKLRDENPKDLDKVALTVLSHSKVSAKNNLILAILKHYQPLCKLSSKVSAIFSTPLQHIVELESKATAKVALQAREILIQGALPSVKERTEQIEHILKSSVVKVAYGSSNPKRSEPDLNILKDLIDSNYVVFDVLLQFLTHQDPVVTAAAAQVYIRRAYRAYTIGDIRVHEGVTVPIVEWKFQLPSAAFSTFPTVKSKMGMNRAVVVSDLSYVANSQSSPLREGILMAVDHLDDVDEILSQSLEVIPRHQSSSNGPAPDRSGSSASLSNVANVCVASTEGFESEEEILVRLREILDLNKQELINASIRRITFMFGFKDGSYPKYYTFNGPNYNENETIRHIEPALAFQLELGRLSNFNIKPIFTDNRNIHVYEAVSKTSPLDKRFFTRGIIRTGHIRDDISIQEYLTSEANRLMSDILDNLEVTDTSNSDLNHIFINFIAVFDISPEDVEAAFGGFLERFGKRLLRLRVSSAEIRIIIKDPQTGAPVPLRALINNVSGYVIKTEMYTEVKNAKGEWVFKSLGKPGSMHLRPIATPYPVKEWLQPKRYKAHLMGTTYVYDFPELFRQASSSQWKNFSADVKLTDDFFISNELIEDENGELTEVEREPGANAIGMVAFKITVKTPEYPRGRQFVVVANDITFKIGSFGPQEDEFFNKVTEYARKRGIPRIYLAANSGARIGMAEEIVPLFQVAWNDAANPDKGFQYLYLTSEGMETLKKFDKENSVLTERTVINGEERFVIKTIIGSEDGLGVECLRGSGLIAGATSRAYHDIFTITLVTCRSVGIGAYLVRLGQRAIQVEGQPIILTGAPAINKMLGREVYTSNLQLGGTQIMYNNGVSHLTAVDDLAGVEKIVEWMSYVPAKRNMPVPILETKDTWDRPVDFTPTNDETYDVRWMIEGRETESGFEYGLFDKGSFFETLSGWAKGVVVGRARLGGIPLGVIGVETRTVENLIPADPANPNSAETLIQEPGQVWHPNSAFKTAQAINDFNNGEQLPMMILANWRGFSGGQRDMFNEVLKYGSFIVDALVDYKQPIIIYIPPTGELRGGSWVVVDPTINADQMEMYADVNARAGVLEPQGMVGIKFRREKLLDTMNRLDDKYRELRSQLSNKSLAPEVHQQISKQLADRERELLPIYGQISLQFADLHDRSSRMVAKGVISKELEWTEARRFFFWRLRRRLNEEYLIKRLSHQVGEASRLEKIARIRSWYPASVDHEDDRQVATWIEENYKTLDDKLKGLKLESFAQDLAKKIRSDHDNAIDGLSEVIKMLSTDDKEKLLKTLK

SEQ ID NO: 15 - protein, 256 aa, Saccharomyces cerevisiae
MDFTKPETVLNLQNIRDELVRMEDSIIFKFIERSHFATCPSVYEANHPGLEIPNFKGSFLDWALSNLEIAHSRIRRFESPDETPFFPDKIQKSFLPSINYPQILAPYAPEVNYNDKIKKVYIEKIIPLISKRDGDDKNNFGSVATRDIECLQSLSRRIHFGKFVAEAKFQSDIPLYTKLIKSKDVEGIMKNITNSAVEEKILERLTKKAEVYGVDPTNESGERRINPEYLVKIYKEIVIPITKEVEVEYLLRRLEE

SEQ ID NO: 16 - protein, 717 aa, Arabidopsis thaliana
MDQIEAMLCGGGEKTKVAVTTKTLADPLNWGLAADQMKGSHLDEVKKMVEEYRRPVVNLGGETLTIGQVAAISTVGGSVKVELAETSRAGVKASSDWVMESMNKGTDSYGVTTGFGATSHRRTKNGTALQTELIRFLNAGIFGNTKETCHTLPQSATRAAMLVRVNTLLQGYSGIRFEILEAITSLLNHNISPSLPLRGTITASGDLVPLSYIAGLLTGRPNSKATGPDGESLTAKEAFEKAGISTGFFDLQPKEGLALVNGTAVGSGMASMVLFEANVQAVLAEVLSAIFAEVMSGKPEFTDHLTHRLKHHPGQIEAAAIMEHILDGSSYMKLAQKVHEMDPLQKPKQDRYALRTSPQWLGPQIEVIRQATKSIEREINSVNDNPLIDVSRNKAIHGGNFQGTPIGVSMDNTRLAIAAIGKLMFAQFSELVNDFYNNGLPSNLTASSNPSLDYGFKGAEIAMASYCSELQYLANPVTSHVQSAEQHNQDVNSLGLISSRKTSEAVDILKLMSTTFLVGICQAVDLRHLEENLRQTVKNTVSQVAKKVLTTGINGELHPSRFCEKDLLKVVDREQVFTYVDDPCSATYPLMQRLRQVIVDHALSNGETEKNAVTSIFQKIGAFEEELKAVLPKEVEAARAAYGNGTAPIPNRIKECRSYPLYRFVREELGTKLLTGEKVVSPGEEFDKVFTAMCEGKLIDPLMDCLKEWNGAPIPIC

SEQ ID NO: 17 - protein, 531 aa, Rhodobacter capsulatus
MTLQSQTAKDCLALDGALTLVQCEAIATHRSRISVTPALRERCARAHARLEHAIAEQRHIYGITTGFGPLANRLIGADQGAELQQNLIYHLATGVGPKLSWAEARALMLARLNSILQGASGASPETIDRIVAVLNAGFAPEVPAQGTVGASGDLTPLAHMVLALQGRGRMIDPSGRVQEAGAVMDRLCGGPLTLAARDGLALVNGTSAMTAIAALTGVEAARAIDAALRHSAVLMEVLSGHAEAWHPAFAELRPHPGQLRATERLAQALDGAGRVCRTLTAARRLTAADLRPEDHPAQDAYSLRVVPQLVGAVWDTLDWHDRVVTCELNSVTDNPIFPEGCAVPALHGGNFMGVHVALASDALNAALVTLAGLVERQIARLTDEKLNKGLPAFLHGGQAGLQSGFMGAQVTATALLAEMRANATPVSVQSLSTNGANQDVVSMGTIAARRARAQLLPLSQIQAILALALAQAMDLLDDPEGQAGWSLTARDLRDRIRAVSPGLRADRPLAGHIEAVAQGLRHPSAAADPPA

SEQ ID NO: 18 - protein, 505 aa, Arabidopsis thaliana
MDLLLLEKSLIAVFVAVILATVISKLRGKKLKLPPGPIPIPIFGNWLQVGDDLNHRNLVDYAKKFGDLFLLRMGQRNLVVVSSPDLTKEVLLTQGVEFGSRTRNVVFDIFTGKGQDMVFTVYGEHWRKMRRIMTVPFFTNKVVQQNREGWEFEAASVVEDVKKNPDSATKGIVLRKRLQLMMYNNMFRIMFDRRFESEDDPLFLRLKALNGERSRLAQSFEYNYGDFIPILRPFLRGYLKICQDVKDRRIALFKKYFVDERKQIASSKPTGSEGLKCAIDHILEAEQKGEINEDNVLYIVENINVAAIETTLWSIEWGIAELVNHPEIQSKLRNELDTVLGPGVQVTEPDLHKLPYLQAVVKETLRLRMAIPLLVPHMNLHDAKLAGYDIPAESKILVNAWWLANNPNSWKKPEEFRPERFFEEESHVEANGNDFRYVPFGVGRRSCPGIILALPILGITIGRMVQNFELLPPPGQSKVDTSEKGGQFSLHILNHSIIVMKPRNC

SEQ ID NO: 19 - protein, 556 aa, Arabidopsis thaliana
MTTQDVIVNDQNDQKQCSNDVIFRSRLPDIYIPNHLPLHDYIFENISEFAAKPCLINGPTGEVYTYADVHVTSRKLAAGLHNLGVKQHDVVMILLPNSPEVVLTFLAASFIGAITTSANPFFTPAEISKQAKASAAKLIVTQSRYVDKIKNLQNDGVLIVTTDSDAIPENCLRFSELTQSEEPRVDSIPEKISPEDVVALPFSSGTTGLPKGVMLTHKGLVTSVAQQVDGENPNLYFNRDDVILCVLPMFHIYALNSIMLCSLRVGATILIMPKFEITLLLEQIQRCKVTVAMVVPPIVLAIAKSPETEKYDLSSVRMVKSGAAPLGKELEDAISAKFPNAKLGQGYGMTEAGPVLAMSLGFAKEPFPVKSGACGTVVRNAEMKILDPDTGDSLPRNKPGEICIRGNQIMKGYLNDPLATASTIDKDGWLHTGDVGFIDDDDELFIVDRLKELIKYKGFQVAPAELESLLIGHPEINDVAVVAMKEEDAGEVPVAFVVRSKDSNISEDEIKQFVSKQVVFYKRINKVFFTDSIPKAPSGKILRKDLRARLANGLMN

SEQ ID NO: 20 - protein, 392 aa, Vitus pseudoreticulata
MASVEEIRNAQRAKGPATILAIGTATPDHCVYQSDYADYYFRVTKSEHMTALKKKFNRICDKSMIKKRYIHLTEEMLEEHPNIGAYMAPSLNIRQEIITAEVPKLGKEAALKALKEWGQPKSKITHLVFCTTSGVEMPGADYKLANLLGLEPSVRRVMLYHQGCYAGGTVLRTTKDLAENNAGARVLVVCPEITVVTFRGPSEDALDSLVGQALFGDGSAAVIVGSDPDISIERPLFQLVSAAQTFIPNFAGAIAGNLREVGLTFHLWPNVPTLISENIENCLTQAFDPLGISDWNSLFWIAHPGGPAILDAVEAKLNLDKKKLEATRHVLSEYGNVSSACVLFILDEMRKKSLKGERATTGEGLGWGVLFGFGPGLTIETVVLHSIPMVTN

SEQ ID NO: 21 - protein, 711 aa, Arabidopsis thaliana
MSSSSSSSTSMIDLMAAIIKGEPVIVSDPANASAYESVAAELSSMLIENRQFAMIVTTSIAVLIGCIVMLVWRRSGSGNSKRVEPLKPLVIKPREEEIDDGRKKVTIFFGTQTGTAEGFAKALGEEAKARYEKTRFKIVDLDDYAADDDEYEEKLKKEDVAFFFLATYGDGEPTDNAARFYKWFTEGNDRGEWLKNLKYGVFGLGNRQYEHFNKVAKVVDDILVEQGAQRLVQVGLGDDDQCIEDDFTAWREALWPELDTILREEGDTAVATPYTAAVLEYRVSIHDSEDAKFNDINMANGNGYTVFDAQHPYKANVAVKRELHTPESDRSCIHLEFDIAGSGLTYETGDHVGVLCDNLSETVDEALRLLDMSPDTYFSLHAEKEDGTPISSSLPPPFPPCNLRTALTRYACLLSSPKKSALVALAAHASDPTEAERLKHLASPAGKDEYSKWVVESQRSLLEVMAEFPSAKPPLGVFFAGVAPRLQPRFYSISSSPKIAETRIHVTCALVYEKMPTGRIHKGVCSTWMKNAVPYEKSENCSSAPIFVRQSNFKLPSDSKVPIIMIGPGTGLAPFRGFLQERLALVESGVELGPSVLFFGCRNRRMDFIYEEELQRFVESGALAELSVAFSREGPTKEYVQHKMMDKASDIWNMISQGAYLYVCGDAKGMARDVHRSLHTIAQEQGSMDSTKAEGFVKNLQTSGRYLRDVW

SEQ ID NO: 22 - protein, 370 aa, Saccharomyces cerevisiae
MSESPMFAANGMPKVNQGAEEDVRILGYDPLASPALLQVQIPATPTSLETAKRGRREAIDIITGKDDRVLVIVGPCSIHDLEAAQEYALRLKKLSDELKGDLSIIMRAYLEKPRTTVGWKGLINDPDVNNTFNINKGLQSARQLFVNLTNIGLPIGSEMLDTISPQYLADLVSFGAIGARTTESQLHRELASGLSFPVGFKNGTDGTLNVAVDACQAAAHSHHFMGVTPHGVAAITTTKGNEHCFVILRGGKKGTNYDAKSVAEAKAQLPAGSNGLMIDYSHGNSNKDFRNQPKVNDVVCEQIANGENAITGVMIESNINEGNQGIPAEGKAGLKYGVSITDACIGWETTEDVLRKLAAAVRQRREVNKK

SEQ ID NO: 23 - protein, 2233 aa, Saccharomyces cerevisiae
MSEESLFESSPQKMEYEITNYSERHTELPGHFIGLNTVDKLEESPLRDFVKSHGGHTVISKILIANNGIAAVKEIRSVRKWAYETFGDDRTVQFVAMATPEDLEANAEYIRMADQYIEVPGGTNNNNYANVDLIVDIAERADVDAVWAGWGHASENPLLPEKLSQSKRKVIFIGPPGNAMRSLGDKISSTIVAQSAKVPCIPWSGTGVDTVHVDEKTGLVSVDDDIYQKGCCTSPEDGLQKAKRIGFPVMIKASEGGGGKGIRQVEREEDFIALYHQAANEIPGSPIFIMKLAGRARHLEVQLLADQYGTNISLFGRDCSVQRRHQKIIEEAPVTIAKAETFHEMEKAAVRLGKLVGYVSAGTVEYLYSHDDGKFYFLELNPRLQVEHPTTEMVSGVNLPAAQLQIAMGIPMHRISDIRTLYGMNPHSASEIDFEFKTQDATKKQRRPIPKGHCTACRITSEDPNDGFKPSGGTLHELNFRSSSNVWGYFSVGNNGNIHSFSDSQFGHIFAFGENRQASRKHMVVALKELSIRGDFRTTVEYLIKLLETEDFEDNTITTGWLDDLITHKMTAEKPDPTLAVICGAATKAFLASEEARHKYIESLQKGQVLSKDLLQTMFPVDFIHEGKRYKFTVAKSGNDRYTLFINGSKCDIILRQLPDGGLLIAIGGKSHTIYWKEEVAATRLSVDSMTTLLEVENDPTQLRTPSPGKLVKFLVENGEHIIKGQPYAEIEVMKMQMPLVSQENGIVQLLKQPGSTIVAGDIMAIMTLDDPSKVKHALPFEGMLPDFGSPVIEGTKPAYKFKSLVSTLENILKGYDNQVIMNASLQQLIEVLRNPKLPYSEWKLHISALHSRLPAKLDEQMEELVARSLRRGAVFPARQLSKLIDMAVKNPEYNPDKLLGAVVEPLADIAHKYSNGLEAHEHSIFVHFLEEYYEVEKLFNGPNVREENIILKLRDENPKDLDKVALTVLSHSKVSAKNNLILAILKHYQPLCKLSSKVSAIFSTPLQHIVELESKATAKVALQAREILIQGALPSVKERTEQIEHILKSSVVKVAYGSSNPKRSEPDLNILKDLIDSNYVVFDVLLQFLTHQDPVVTAAAAQVYIRRAYRAYTIGDIRVHEGVTVPIVEWKFQLPSAAFSTFPTVKSKMGMNRAVSVSDLSYVANSQSSPLREGILMAVDHLDDVDEILSQSLEVIPRHQSSSNGPAPDRSGSSASLSNVANVCVASTEGFESEEEILVRLREILDLNKQELINASIRRITFMFGFKDGSYPKYYTFNGPNYNENETIRHIEPALAFQLELGRLSNFNIKPIFTDNRNIHVYEAVSKTSPLDKRFFTRGIIRTGHIRDDISIQEYLTSEANRLMSDILDNLEVTDTSNSDLNHIFINFIAVFDISPEDVEAAFGGFLERFGKRLLRLRVSSAEIRIIIKDPQTGAPVPLRALINNVSGYVIKTEMYTEVKNAKGEWVFKSLGKPGSMHLRPIATPYPVKEWLQPKRYKAHLMGTTYVYDFPELFRQASSSQWKNFSADVKLTDDFFISNELIEDENGELTEVEREPGANAIGMVAFKITVKTPEYPRGRQFVVVANDITFKIGSFGPQEDEFFNKVTEYARKRGIPRIYLAANSGARIGMAEEIVPLFQVAWNDAANPDKGFQYLYLTSEGMETLKKFDKENSVLTERTVINGEERFVIKTIIGSEDGLGVECLRGSGLIAGATSRAYHDIFTITLVTCRSVGIGAYLVRLGQRAIQVEGQPIILTGAPAINKMLGREVYTSNLQLGGTQIMYNNGVSHLTAVDDLAGVEKIVEWMSYVPAKRNMPVPILETKDTWDRPVDFTPTNDETYDVRWMIEGRETESGFEYGLFDKGSFFETLSGWAKGVVVGRARLGGIPLGVIGVETRTVENLIPADPANPNSAETLIQEPGQVWHPNSAFKTAQAINDFNNGEQLPMMILANWRGFSGGQRDMFNEVLKYGSFIVDALVDYKQPIIIYIPPTGELRGGSWVVVDPTINADQMEMYADVNARAGVLEPQGMVGIKFRREKLLDTMNRLDDKYRELRSQLSNKSLAPEVHQQISKQLADRERELLPIYGQISLQFADLHDRSSRMVAKGVISKELEWTEARRFFFWRLRRRLNEEYLIKRLSHQVGEASRLEKIARIRSWYPASVDHEDDRQVATWIEENYKTLDDKLKGLKLESFAQDLAKKIRSDHDNAIDGLSEVIKMLSTDDKEKLLKTLK

SEQ ID NO: 24 - protein, 2233 aa, Saccharomyces cerevisiae
MSEESLFESSPQKMEYEITNYSERHTELPGHFIGLNTVDKLEESPLRDFVKSHGGHTVISKILIANNGIAAVKEIRSVRKWAYETFGDDRTVQFVAMATPEDLEANAEYIRMADQYIEVPGGTNNNNYANVDLIVDIAERADVDAVWAGWGHASENPLLPEKLSQSKRKVIFIGPPGNAMRSLGDKISSTIVAQSAKVPCIPWSGTGVDTVHVDEKTGLVSVDDDIYQKGCCTSPEDGLQKAKRIGFPVMIKASEGGGGKGIRQVEREEDFIALYHQAANEIPGSPIFIMKLAGRARHLEVQLLADQYGTNISLFGRDCSVQRRHQKIIEEAPVTIAKAETFHEMEKAAVRLGKLVGYVSAGTVEYLYSHDDGKFYFLELNPRLQVEHPTTEMVSGVNLPAAQLQIAMGIPMHRISDIRTLYGMNPHSASEIDFEFKTQDATKKQRRPIPKGHCTACRITSEDPNDGFKPSGGTLHELNFRSSSNVWGYFSVGNNGNIHSFSDSQFGHIFAFGENRQASRKHMVVALKELSIRGDFRTTVEYLIKLLETEDFEDNTITTGWLDDLITHKMTAEKPDPTLAVICGAATKAFLASEEARHKYIESLQKGQVLSKDLLQTMFPVDFIHEGKRYKFTVAKSGNDRYTLFINGSKCDIILRQLSDGGLLIAIGGKSHTIYWKEEVAATRLSVDSMTTLLEVENDPTQLRTPSPGKLVKFLVENGEHIIKGQPYAEIEVMKMQMPLVSQENGIVQLLKQPGSTIVAGDIMAIMTLDDPSKVKHALPFEGMLPDFGSPVIEGTKPAYKFKSLVSTLENILKGYDNQVIMNASLQQLIEVLRNPKLPYSEWKLHISALHSRLPAKLDEQMEELVARSLRRGAVFPARQLSKLIDMAVKNPEYNPDKLLGAVVEPLADIAHKYSNGLEAHEHSIFVHFLEEYYEVEKLFNGPNVREENIILKLRDENPKDLDKVALTVLSHSKVSAKNNLILAILKHYQPLCKLSSKVSAIFSTPLQHIVELESKATAKVALQAREILIQGALPSVKERTEQIEHILKSSVVKVAYGSSNPKRSEPDLNILKDLIDSNYVVFDVLLQFLTHQDPVVTAAAAQVYIRRAYRAYTIGDIRVHEGVTVPIVEWKFQLPSAAFSTFPTVKSKMGMNRAVPVSDLSYVANSQSSPLREGILMAVDHLDDVDEILSQSLEVIPRHQSSSNGPAPDRSGSSASLSNVANVCVASTEGFESEEEILVRLREILDLNKQELINASIRRITFMFGFKDGSYPKYYTFNGPNYNENETIRHIEPALAFQLELGRLSNFNIKPIFTDNRNIHVYEAVSKTSPLDKRFFTRGIIRTGHIRDDISIQEYLTSEANRLMSDILDNLEVTDTSNSDLNHIFINFIAVFDISPEDVEAAFGGFLERFGKRLLRLRVSSAEIRIIIKDPQTGAPVPLRALINNVSGYVIKTEMYTEVKNAKGEWVFKSLGKPGSMHLRPIATPYPVKEWLQPKRYKAHLMGTTYVYDFPELFRQASSSQWKNFSADVKLTDDFFISNELIEDENGELTEVEREPGANAIGMVAFKITVKTPEYPRGRQFVVVANDITFKIGSFGPQEDEFFNKVTEYARKRGIPRIYLAANSGARIGMAEEIVPLFQVAWNDAANPDKGFQYLYLTSEGMETLKKFDKENSVLTERTVINGEERFVIKTIIGSEDGLGVECLRGSGLIAGATSRAYHDIFTITLVTCRSVGIGAYLVRLGQRAIQVEGQPIILTGAPAINKMLGREVYTSNLQLGGTQIMYNNGVSHLTAVDDLAGVEKIVEWMSYVPAKRNMPVPILETKDTWDRPVDFTPTNDETYDVRWMIEGRETESGFEYGLFDKGSFFETLSGWAKGVVVGRARLGGIPLGVIGVETRTVENLIPADPANPNSAETLIQEPGQVWHPNSAFKTAQAINDFNNGEQLPMMILANWRGFSGGQRDMFNEVLKYGSFIVDALVDYKQPIIIYIPPTGELRGGSWVVVDPTINADQMEMYADVNARAGVLEPQGMVGIKFRREKLLDTMNRLDDKYRELRSQLSNKSLAPEVHQQISKQLADRERELLPIYGQISLQFADLHDRSSRMVAKGVISKELEWTEARRFFFWRLRRRLNEEYLIKRLSHQVGEASRLEKIARIRSWYPASVDHEDDRQVATWIEENYKTLDDKLKGLKLESFAQDLAKKIRSDHDNAIDGLSEVIKMLSTDDKEKLLKTLK

SEQ ID NO: 25 - protein, 2233 aa, Saccharomyces cerevisiae
MSEESLFESSPQKMEYEITNYSERHTELPGHFIGLNTVDKLEESPLRDFVKSHGGHTVISKILIANNGIAAVKEIRSVRKWAYETFGDDRTVQFVAMATPEDLEANAEYIRMADQYIEVPGGTNNNNYANVDLIVDIAERADVDAVWAGWGHASENPLLPEKLSQSKRKVIFIGPPGNAMRSLGDKISSTIVAQSAKVPCIPWSGTGVDTVHVDEKTGLVSVDDDIYQKGCCTSPEDGLQKAKRIGFPVMIKASEGGGGKGIRQVEREEDFIALYHQAANEIPGSPIFIMKLAGRARHLEVQLLADQYGTNISLFGRDCSVQRRHQKIIEEAPVTIAKAETFHEMEKAAVRLGKLVGYVSAGTVEYLYSHDDGKFYFLELNPRLQVEHPTTEMVSGVNLPAAQLQIAMGIPMHRISDIRTLYGMNPHSASEIDFEFKTQDATKKQRRPIPKGHCTACRITSEDPNDGFKPSGGTLHELNFRSSSNVWGYFSVGNNGNIHSFSDSQFGHIFAFGENRQASRKHMVVALKELSIRGDFRTTVEYLIKLLETEDFEDNTITTGWLDDLITHKMTAEKPDPTLAVICGAATKAFLASEEARHKYIESLQKGQVLSKDLLQTMFPVDFIHEGKRYKFTVAKSGNDRYTLFINGSKCDIILRQLPDGGLLIAIGGKSHTIYWKEEVAATRLSVDSMTTLLEVENDPTQLRTPSPGKLVKFLVENGEHIIKGQPYAEIEVMKMQMPLVSQENGIVQLLKQPGSTIVAGDIMAIMTLDDPSKVKHALPFEGMLPDFGSPVIEGTKPAYKFKSLVSTLENILKGYDNQVIMNASLQQLIEVLRNPKLPYSEWKLHISALHSRLPAKLDEQMEELVARSLRRGAVFPARQLSKLIDMAVKNPEYNPDKLLGAVVEPLADIAHKYSNGLEAHEHSIFVHFLEEYYEVEKLFNGPNVREENIILKLRDENPKDLDKVALTVLSHSKVSAKNNLILAILKHYQPLCKLSSKVSAIFSTPLQHIVELESKATAKVALQAREILIQGALPSVKERTEQIEHILKSSVVKVAYGSSNPKRSEPDLNILKDLIDSNYVVFDVLLQFLTHQDPVVTAAAAQVYIRRAYRAYTIGDIRVHEGVTVPIVEWKFQLPSAAFSTFPTVKSKMGMNRAVPVSDLSYVANSQSSPLREGILMAVDHLDDVDEILSQSLEVIPRHQSSSNGPAPDRSGSSASLSNVANVCVASTEGFESEEEILVRLREILDLNKQELINASIRRITFMFGFKDGSYPKYYTFNGPNYNENETIRHIEPALAFQLELGRLSNFNIKPIFTDNRNIHVYEAVSKTSPLDKRFFTRGIIRTGHIRDDISIQEYLTSEANRLMSDILDNLEVTDTSNSDLNHIFINFIAVFDISPEDVEAAFGGFLERFGKRLLRLRVSSAEIRIIIKDPQTGAPVPLRALINNVSGYVIKTEMYTEVKNAKGEWVFKSLGKPGSMHLRPIATPYPVKEWLQPKRYKAHLMGTTYVYDFPELFRQASSSQWKNFSADVKLTDDFFISNELIEDENGELTEVEREPGANAIGMVAFKITVKTPEYPRGRQFVVVANDITFKIGSFGPQEDEFFNKVTEYARKRGIPRIYLAANSGARIGMAEEIVPLFQVAWNDAANPDKGFQYLYLTSEGMETLKKFDKENSVLTERTVINGEERFVIKTIIGSEDGLGVECLRGSGLIAGATSRAYHDIFTITLVTCRSVGIGAYLVRLGQRAIQVEGQPIILTGAPAINKMLGREVYTSNLQLGGTQIMYNNGVSHLTAVDDLAGVEKIVEWMSYVPAKRNMPVPILETKDTWDRPVDFTPTNDETYDVRWMIEGRETESGFEYGLFDKGSFFETLSGWAKGVVVGRARLGGIPLGVIGVETRTVENLIPADPANPNSAETLIQEPGQVWHPNSAFKTAQAINDFNNGEQLPMMILANWRGFSGGQRDMFNEVLKYGSFIVDALVDYKQPIIIYIPPTGELRGGSWVVVDPTINADQMEMYADVNARAGVLEPQGMVGIKFRREKLLDTMNRLDDKYRELRSQLSNKSLAPEVHQQISKQLADRERELLPIYGQISLQFADLHDRSSRMVAKGVISKELEWTEARRFFFWRLRRRLNEEYLIKRLSHQVGEASRLEKIARIRSWYPASVDHEDDRQVATWIEENYKTLDDKLKGLKLESFAQDLAKKIRSDHDNAIDGLSEVIKMLSTDDKEKLLKTLK

SEQ ID NO: 26 - DNA, 420 bp, Artificial Sequence
  pTEF1 promoter
gcacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaattacaa

(19) United States
(12) Patent Application Publication
(10) Pub. No.: US 2018/0155733 A1
(43) Pub. Date: Jun. 7, 2018

(54) A METHOD FOR PRODUCING RESVERATROL

(71) Applicant: EVOLVA SA, Reinach (CH)

(72) Inventors: Ernesto SIMON VECILLA, Glostrup (DK); Carlos Casado VAZQUEZ, Copenhagen (DK); Niels Bjerg JENSEN, Reinach (CH)

(21) Appl. No.: 15/572,937

(22) PCT Filed: May 12, 2016
(86) PCT No.: PCT/EP2016/060798

§ 371 Date: Nov. 9, 2017

Related U.S. Application Data
(60) Provisional application No. 62/160,505, filed on May 12, 2015.

(51) Int. Cl. C12N 15/81; C12P 7/22

(52) U.S. Cl. CPC C12N 15/81; C12P 7/22; C12Y 504/99005; C12Y 207/11027; C12Y 205/01054

(57) ABSTRACT

Recombinant hosts and methods for producing resveratrol in recombinant hosts are disclosed herein.

### BACKGROUND OF THE INVENTION

### Field of the Invention

The invention disclosed herein relates generally to the fields of genetic engineering. Particularly, the invention disclosed herein provides methods for producing increased yields of stilbenes and flavonoids in a genetically modified cell.

### Description of Related Art

Flavonoids and stilbenes are two groups of secondary plant metabolites derived from the phenylpropanoid pathway by different routes. Both flavonoids and stilbenes are recognized as having antifungal and antibacterial properties in plants. Flavonoids have also been investigated for application to several human pathological conditions, including cancer, diabetes, obesity and Parkinson's disease (see e.g., Koopman et al., 2012, Microbial Cell Factories 11:155; Wang & Zhang, 2012, Scanning 34: 1-5; Zava & Duwe, 1997, Nutr. Cancer 27: 31-40; Greenwald, 2004, J Nutr 134: 3507S-3512S; Hou et al., 2004, J Biomed Biotechnol 2004: 321-325; Allister et al., 2005, Diabetes 54: 1676-1683). Flavonoids have been shown to act as scavengers of oxygen radicals, and may possess anti-inflammatory, antiviral and anti-tumor activities (see e.g., Koopman et al., 2012, Microbial Cell Factories 11:155; Nijveldt et al., 2001, Am J Clin Nutr 74: 418-425; Limem et al., 2008, Process Biochem 43:463-479). Currently, flavonoid production is mainly achieved by isolation from plants. Production of flavonoids is inefficient, however, due primarily to low growth rate of producing plants coupled with complex extraction and separation of flavonoids from related structures.

Resveratrol (3,5,4′-trihydroxy-stilbene) is a phytophenol belonging to the group of stilbene phytoalexins, which are low-molecular-mass secondary metabolites that constitute the active defense mechanism in plants in response to fungal and other infections or other stress-related events (see, e.g., WO2006089898 (A1)). In addition to its antifungal properties, resveratrol has been recognized for its cardioprotective and cancer chemopreventive activities in humans; it acts as a phytoestrogen, an inhibitor of platelet aggregation (Kopp et al., 1998, European J Endocrinol. 138: 619-620; Gehm et al., 1997, Proc Natl Acad Sci USA 94: 14138-14143; Lobo et al., 1995, Am. J. Obstet. Gynecol. 173: 982-989; Gao & Ming, 2010, Mini Rev Med Chem 10(6):550-67), and an antioxidant (Tang et al., 1997, Science 275: 218-220; Huang, 1997, Food Sci. 24: 713-727).

Plants, the skin of red grapes, and other fruits produce resveratrol naturally. Present production processes rely mostly upon extraction of resveratrol, either from the skin of grape berries or from the plant Fallopia japonica or Polygonum cuspidatum, known as “Japanese knotweed.” Current extraction and purification methods use organic solvents to extract resveratrol and separate it from the biomass and/or cell debris. Examples of these solvents include, among others, ethanol, methanol, ethyl acetate, and petroleum ether. This is a labor-intensive process and generates low yields. Moreover, since resveratrol has low water-solubility (see, e.g., Gao & Ming, 2010, Mini Rev Med Chem 10(6):550-67), it forms aggregates/crystals upon addition to water and/or formation by a recombinant resveratrol producing- and secreting-microorganism. Separation of resveratrol aggregates/crystals from recombinant or other cells (such as microorganisms or plant cells) by centrifugation is inefficient.

Generally, stilbenes, including resveratrol, and flavonoids are produced in plants and yeast through the phenylpropanoid pathway as illustrated by the reactions shown in FIGS. 1 and 2 and as described in WO2006089898 (A1), which is incorporated by reference in its entirety herein. In yeast, the starting metabolites are malonyl-CoA and phenylalanine or tyrosine. The amino acid L-phenylalanine is converted into trans-cinnamic acid through non-oxidative deamination by L-phenylalanine ammonia lyase (PAL). Next, trans-cinnamic acid is hydroxylated at the para-position to 4-coumaric acid (4-hydroxycinnamic acid) by cinnamate-4-hydroxylase (C4H), a cytochrome P450 monooxygenase enzyme, in conjunction with NADPH:cytochrome P450 reductase (CPR). Alternatively, the amino acid L-tyrosine is converted into 4-coumaric acid by tyrosine ammonia lyase (TAL). The 4-coumaric acid from either alternative pathway is subsequently activated to 4-coumaroyl-CoA by the action of 4-coumarate-CoA ligase (4CL). Within the phenylpropanoid pathway, 4-coumaroyl-CoA represents the key branching point from which flavonoids and stilbenes are derived. Stilbenes are synthesized via stilbene synthase (STS), also known as resveratrol synthase (RS), catalyzing condensation of a phenylpropane unit of 4-coumaroyl-CoA with malonyl-CoA, resulting in formation of resveratrol. Conversely, the first step in flavonoid synthesis is condensation of a phenylpropane unit of 4-coumaroyl-CoA with malonyl-CoA and chalcone synthase (CHS), resulting in the formation of tetrahydroxychalcone.

Previously, a yeast strain was disclosed that could produce resveratrol from 4-coumaric acid that is found in small quantities in grape must (Becker et al., 2003, FEMS Yeast Res. 4: 79-85). Production of 4-coumaroyl-CoA, and concomitantly resveratrol, in laboratory strains of Saccharomyces cerevisiae, has been achieved by co-expressing a heterologous coenzyme-A ligase gene from hybrid poplar, together with the grapevine resveratrol synthase gene (vst1) (Becker et al., 2003, FEMS Yeast Res. 4: 79-85). Another substrate for resveratrol synthase, malonyl-CoA, is endogenously produced in yeast. Becker et al., 2003, Id., indicated that S. cerevisiae cells produced minute amounts of resveratrol in the piceid form when cultured in synthetic media supplemented with 4-coumaric acid. However, said yeast strain would not be suitable for commercial application because it results in low resveratrol yields and requires the addition of 4-coumaric acid, which is expensive and not often present in industrial media. Therefore, there remains a need for an in vivo expression system that produces high yields of resveratrol more economically.

### SUMMARY OF THE INVENTION

It is against the above background that the present invention provides certain advantages and advancements over the prior art.

Although this invention disclosed herein is not limited to specific advantages or functionality, the invention disclosed herein provides a recombinant host comprising:
(a) a gene encoding a 3-Deoxy-D-arabinoheptulosonate 7-phosphate (DAHP) synthase polypeptide;
(b) a gene encoding a chorismate mutase polypeptide; and
(c) a gene encoding an acetyl-CoA carboxylase polypeptide;
wherein at least one of the genes is a recombinant gene,
wherein the host is capable of producing a stilbene.

The invention further provides a method of producing a stilbene, comprising:
(a) growing a recombinant host in a culture medium, under conditions in which the genes are expressed,
   wherein the host comprises:
   (i) a gene encoding a 3-Deoxy-D-arabinoheptulosonate 7-phosphate (DAHP) synthase polypeptide;
   (ii) a gene encoding a chorismate mutase polypeptide; and
   (iii) a gene encoding an acetyl-CoA carboxylase polypeptide;
   wherein at least one of the genes is a recombinant gene, and, optionally
(b) recovering the stilbene from the culture media.

In some aspects, the DAHP synthase polypeptide comprises a tyrosine-sensitive 3-Deoxy-D-arabinoheptulosonate 7-phosphate synthase (ARO4) polypeptide having at least 65% identity to the amino acid sequence set forth in SEQ ID NO: 1.

In some aspects, the DAHP synthase polypeptide comprises an ARO4 polypeptide having a mutation at K229.

In some aspects, the DAHP synthase polypeptide comprises an ARO4 polypeptide having a mutation that is K229L.

In some aspects, the DAHP synthase polypeptide is not feedback inhibited.

In some aspects of the recombinant host or methods disclosed herein, the recombinant host further comprises disruption of a gene encoding a phenylalanine-inhibited 3-Deoxy-D-arabinoheptulosonate 7-phosphate synthase (ARO3) polypeptide, whereby the host does not express ARO3.

In some aspects, the ARO3 polypeptide has at least 75% identity to the amino acid sequence set forth in SEQ ID NO: 3.

In some aspects, the chorismate mutase polypeptide comprises a chorismate mutase (ARO7) polypeptide having at least 50% identity to the amino acid sequence set forth in SEQ ID NO: 4.

In some aspects, the chorismate mutase polypeptide comprises an ARO7 polypeptide having a mutation at T226.

In some aspects, the chorismate mutase polypeptide comprises an ARO7 polypeptide having a mutation that is T226I or T226N.

In some aspects, the chorismate mutase polypeptide is not feedback inhibited.

In some aspects, the acetyl-CoA carboxylase polypeptide comprises an acetyl-CoA carboxylase alpha (ACC1) polypeptide having at least 70% identity to the amino acid sequence set forth in SEQ ID NO: 6.

In some aspects, the acetyl-CoA carboxylase polypeptide comprises an ACC1 polypeptide having at least one mutation at S659 or S1157.

In some aspects, the acetyl-CoA carboxylase polypeptide comprises an ACC1 polypeptide having at least one mutation at S659 or S1157, wherein the replacement amino acid is non-phosphorylable.

In some aspects, the acetyl-CoA carboxylase polypeptide comprises an ACC1 polypeptide having at least one mutation that is S659A, S659V, S1157A, or S1157V.

In some aspects, the acetyl-CoA carboxylase polypeptide is not feedback inhibited.

In some embodiments of the recombinant host or methods disclosed herein, the recombinant host further comprises one or more of:
(a) a gene encoding a L-phenylalanine ammonia lyase (PAL) polypeptide;
(b) a gene encoding a cinnamate-4-hydroxylase (C4H) polypeptide;
(c) a gene encoding a NADPH:cytochrome P450 reductase polypeptide;
(d) a gene encoding a tyrosine ammonia lyase (TAL) polypeptide;
(e) a gene encoding a 4-coumarate-CoA ligase (4CL) polypeptide; or
(f) a gene encoding a stilbene synthase (STS) polypeptide;
wherein at least one of the genes is a recombinant gene.

In some aspects of the recombinant host or methods disclosed herein, the stilbene produced by the recombinant host is resveratrol or a resveratrol derivative.

In some aspects of the recombinant host or methods disclosed herein, the recombinant host comprises a microorganism that is a yeast cell, a plant cell, a mammalian cell, an insect cell, a fungal cell, or a bacterial cell.

In some aspects of the recombinant host or methods disclosed herein, the bacterial cell comprises Escherichia bacteria cells, Lactobacillus bacteria cells, Lactococcus bacteria cells, Cornebacterium bacteria cells, Acetobacter bacteria cells, Acinetobacter bacteria cells, or Pseudomonas bacterial cells.

In some aspects of the recombinant host or methods disclosed herein, the yeast cell is a cell from Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactic, Hansenula polymorpha, Candida boidinii, Arxula adeninivorans, Xanthophyllomyces dendrorhous, or Candida albicans species.

In some aspects of the recombinant host or methods disclosed herein, the yeast cell is a Saccharomycete.

In some aspects of the recombinant host or methods disclosed herein, the yeast cell is a cell from the Saccharomyces cerevisiae species.

In some aspects, the methods disclosed herein further comprise the step of detecting the recovered stilbene by thin layer chromatography (TLC), high-performance liquid chromatography (HPLC), ultraviolet-visible spectroscopy/spectrophotometry (UV-Vis), liquid chromatography-mass spectrometry (LC-MS), or nuclear magnetic resonance (NMR).

The invention disclosed herein also provides a recombinant host comprising one or more of:

(a) a gene encoding a 3-Deoxy-D-arabinoheptulosonate 7-phosphate (DAHP) synthase polypeptide;

(b) a gene encoding a chorismate mutase polypeptide; and

(c) a gene encoding an acetyl-CoA carboxylase polypeptide;

wherein at least one of the genes is a recombinant gene,

wherein the host is capable of producing a flavonoid compound.

The invention disclosed herein also provides a method of producing a flavonoid compound, comprising:

(a) growing a recombinant host in a culture medium, under conditions in which the host produces a flavonoid,

wherein the host comprises one or more of:
(i) a gene encoding a 3-Deoxy-D-arabinoheptulosonate 7-phosphate (DAHP) synthase polypeptide;
(ii) a gene encoding a chorismate mutase polypeptide; and
(iii) a gene encoding an acetyl-CoA carboxylase polypeptide;
wherein at least one of the genes is a recombinant gene,

and, optionally

(b) recovering the flavonoid from the culture media.

In some aspects of the recombinant host or methods disclosed herein, the recombinant host further comprises one or more of:

(a) a gene encoding a L-phenylalanine ammonia lyase (PAL) polypeptide;

(b) a gene encoding a cinnamate-4-hydroxylase (C4H) polypeptide;

(c) a gene encoding a NADPH:cytochrome P450 reductase polypeptide;

(d) a gene encoding a tyrosine ammonia lyase (TAL) polypeptide;

(e) a gene encoding a 4-coumarate-CoA ligase (4CL) polypeptide; or

(f) a gene encoding a chalcone synthase (CHS) polypeptide;

wherein at least one of said genes is a recombinant gene.

These and other features and advantages of the present invention will be more fully understood from the following detailed description of the invention taken together with the accompanying claims. It is noted that the scope of the claims is defined by the recitations therein and not by the specific discussion of features and advantages set forth in the present description.

### BRIEF DESCRIPTION OF THE DRAWINGS

The following detailed description of the embodiments of the present invention can be best understood when read in conjunction with the following drawings, where like structure is indicated with like reference numerals and in which:

FIG. 1 shows a schematic diagram of the resveratrol pathway from L-phenylalanine or L-tyrosine in plants and yeast.

FIG. 2 shows a schematic diagram of a pathway for producing resveratrol from glucose in yeast.

FIG. 3A shows a schematic diagram of a pathway for producing phenylpyruvate and hydroxyphenlpyruvate from phosphoenolpyruvate and erythrose 4-phosphate in an S. cerevisiae strain overexpressing tyrosine-sensitive 3-Deoxy-D-arabinoheptulosonate 7-phosphate synthase (ARO4) and chorismate mutase (ARO7) and deleted of phenylalanine-inhibited 3-Deoxy-D-arabinoheptulosonate 7-phosphate synthase (ARO3).

FIG. 3B shows a schematic diagram of a pathway for producing malonyl-CoA from acetyl-CoA in an S. cerevisiae strain overexpressing acetyl-CoA carboxylase alpha (ACC1).

FIG. 4A shows the amino acid sequence of S. cerevisiae ARO4 (SEQ ID NO:1).

FIG. 4B shows amino acid sequence of the K229L mutant of S. cerevisiae ARO4 (SEQ ID NO:2).

FIG. 4C shows the amino acid sequence of S. cerevisiae ARO3 (SEQ ID NO:3).

FIG. 4D shows the amino acid sequence of S. cerevisiae ARO7 (SEQ ID NO:4).

FIG. 4E shows the amino acid sequence of the T226I mutant of S. cerevisiae ARO7 (SEQ ID NO:5).

FIG. 4F shows S. cerevisiae ACC1 (SEQ ID N0:6).

FIG. 4G shows the amino acid sequence of the S659A mutant of S. cerevisiae ACC1 (SEQ ID NO:7).

FIG. 4H shows the amino acid sequence of the S659V mutant of S. cerevisiae ACC1 (SEQ ID NO:8).

FIG. 4I shows the amino acid sequence of the S1157A mutant of S. cerevisiae ACC1 (SEQ ID NO:9).

FIG. 4J shows the amino acid sequence of the S1157V mutant of S. cerevisiae ACC1 (SEQ ID NO:10).

FIG. 4K shows the amino acid sequence of the S659A S1157A mutant of S. cerevisiae ACC1 (SEQ ID NO:11).

FIG. 4L shows the amino acid sequence of the S659A S1157V mutant of S. cerevisiae ACC1 (SEQ ID NO:12).

FIG. 4M shows the amino acid sequence of the S659V S1157A mutant of S. cerevisiae ACC1 (SEQ ID NO:13).

FIG. 4N shows the amino acid sequence of the S659V S1157V mutant of S. cerevisiae ACC1 (SEQ ID NO:14).

FIG. 4O shows the amino acid sequence of the T226N mutant of S. cerevisiae ARO7 (SEQ ID NO:15).

FIG. 4P shows the amino acid sequence of A. thaliana PAL2 (SEQ ID NO:16).

FIG. 4Q shows the amino acid sequence of R. capsulatus TAL (SEQ ID NO:17).

FIG. 4R shows the amino acid sequence of A. thaliana C4H (SEQ ID NO:18).

FIG. 4S shows the amino acid sequence of A. thaliana 4CL2 (SEQ ID NO:19).

FIG. 4T shows the amino acid sequence of V. pseudoreticulata STS (SEQ ID NO:20).

FIG. 4U shows the amino acid sequence of A. thaliana ATR2 (SEQ ID NO:21).

FIG. 4V shows the amino acid sequence of the K229P mutant of S. cerevisiae ARO4 (SEQ ID NO:22).

FIG. 4W shows the amino acid sequence of the S659P mutant of S. cerevisiae ACC1 (SEQ ID NO:23).

FIG. 4X shows the amino acid sequence of the S1157P mutant of S. cerevisiae ACC1 (SEQ ID NO:24).

FIG. 4Y shows the amino acid sequence of the S659P S1157P mutant of S. cerevisiae ACC1 (SEQ ID NO:25).

FIG. 4Z shows the nucleic acid sequence of the pTEF1 promoter (SEQ ID NO:26).

FIG. 5 shows production of resveratrol and secondary metabolites in a yeast strain transformed with empty plasmid (control strain), a yeast strain overexpressing mutant S. cerevisiae ARO4 K229L and ARO7 T226I genes (ARO4*-ARO7*), a yeast strain overexpressing mutant S. cerevisiae ARO4 K229L gene (ARO4*), an ARO3 knockout yeast strain overexpressing mutant S. cerevisiae ARO4 K229L and ARO7 T226I genes (aro3::ARO4*-ARO7*), and an ARO3 knockout yeast strain overexpressing mutant S. cerevisiae ARO4 K229L gene (aro3::ARO4*). Secondary metabolites are likely produced due to conversion of starting metabolites malonyl-CoA, phenylalanine and/or tyrosine into intermediates (e.g., coumaric acid) and side products (e.g., phloretic acid), rather than resveratrol.

FIG. 6 shows resveratrol titers over time of fed-batch fermentation in an ARO3 knockout yeast strain overexpressing mutant S. cerevisiae ARO4 K229L and ARO7 T226I genes (aro3::ARO4*-ARO7*) and a reference strain in which the same strain above (aro3::ARO4*-ARO7*) was further transformed with a URA3 selection marker deleting the overexpressed ARO4 K229L and ARO7 T226I genes (aro3::URA3).

FIG. 7 shows total titer of resveratrol and secondary metabolites over time of fed-batch fermentation in an ARO3 knockout yeast strain overexpressing mutant S. cerevisiae ARO4 K229L and ARO7 T226I genes (aro3::ARO4*-ARO7*) and a reference strain in which the same strain above (aro3::ARO4*-ARO7*) was further transformed with a URA3 selection marker deleting the overexpressed ARO4 K229L and ARO7 T226I genes (aro3::URA3). Secondary metabolites are likely produced due to conversion of starting metabolites malonyl-CoA, phenylalanine and/or tyrosine into intermediates (e.g., coumaric acid) and side products (e.g., phloretic acid), rather than resveratrol.

FIG. 8 shows resveratrol titer during fed-batch cultivation of resveratrol-producing strains. Titers are expressed as improvement relative to the titer at 140 hours of the strain overexpressing only the endogenous, not mutated, ACC1 due to promoter replacement with the strong constitutive promoter pTEF1.

Skilled artisans will appreciate that elements in the Figures are illustrated for simplicity and clarity and have not necessarily been drawn to scale. For example, the dimensions of some of the elements in the Figures can be exaggerated relative to other elements to help improve understanding of the embodiment(s) of the present invention.

### DETAILED DESCRIPTION OF THE INVENTION

All publications, patents and patent applications cited herein are hereby expressly incorporated by reference for all purposes.

Methods well known to those skilled in the art can be used to construct genetic expression constructs and recombinant cells according to this invention. These methods include in vitro recombinant DNA techniques, synthetic techniques, in vivo recombination techniques, and PCR techniques. See, for example, techniques as described in Maniatis et al., 1989, MOLECULAR CLONING: A LABORATORY MANUAL, Cold Spring Harbor Laboratory, New York; Ausubel et al., 1989, CURRENT PROTOCOLS IN MOLECULAR BIOLOGY, Greene Publishing Associates and Wiley Interscience, New York, and PCR Protocols: A Guide to Methods and Applications (Innis et al., 1990, Academic Press, San Diego, Calif.).

Before describing the present invention in detail, a number of terms will be defined. As used herein, the singular forms “a”, “an”, and “the” include plural referents unless the context clearly dictates otherwise. For example, reference to a “nucleic acid” means one or more nucleic acids.

It is noted that terms like “preferably”, “commonly”, and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

For the purposes of describing and defining the present invention it is noted that the terms “increase”, “increases”, “increased”, “greater”, ‘higher”, and “lower” are utilized herein to represent non-quantitative comparisons, values, measurements, or other representations to a stated reference or control.

For the purposes of describing and defining the present invention it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

As used herein, the terms “polynucleotide”, “nucleotide”, “oligonucleotide”, and “nucleic acid” can be used interchangeably to refer to nucleic acid comprising DNA, RNA, derivatives thereof, or combinations thereof.

As used herein, the terms “feedback-inhibited” and “feedback inhibited” are used interchangeably to refer to deactivation of a stated enzyme by a product of the reaction catalyzed by the same enzyme once the product reaches a threshold level.

As used herein, the term “tyrosine-sensitive” refers to an enzyme which can be feedback-inhibited by tyrosine.

### Production of Resveratrol or Modified Resveratrol

Resveratrol can be synthesized in vitro, by bioconversion, or in a recombinant host. As used herein, the term “recombinant host” is intended to refer to a host cell, the genome of which has been augmented by at least one incorporated DNA sequence. Such DNA sequences include, but are not limited to, genes that are not naturally present, DNA sequences that are not normally transcribed into RNA or translated into a protein (“expressed”), and other genes or DNA sequences that are desired to be introduced into the cell to produce the recombinant host. It will be appreciated that the genome of a recombinant host described herein is typically augmented through stable introduction of one or more recombinant genes. Generally, the introduced DNA is not originally resident in the host that is the recipient of the DNA, but it is within the scope of the invention to isolate a DNA segment from a given host, and to subsequently introduce one or more additional copies of that DNA into the same host, e.g., to enhance production of the product of a gene or alter the expression pattern of a gene. In some instances, the introduced DNA will modify or replace an endogenous gene or DNA sequence by, e.g., homologous recombination or site-directed mutagenesis. Suitable recombinant hosts include microorganisms, plant cells, and plants.

The term “recombinant gene” refers to a gene or DNA sequence that is introduced into a recipient host, regardless of whether the same or a similar gene or DNA sequence may already be present in such a host. “Introduced” or “augmented” in this context is known in the art to mean introduced or augmented by the hand of man. Thus, a recombinant gene may be a DNA sequence from another species, or may be a DNA sequence that originated from or is present in the same species, but has been incorporated into a host by recombinant methods to form a recombinant host. It will be appreciated that a recombinant gene that is introduced into a host can be identical to a DNA sequence that is normally present in the host being transformed and is introduced to provide one or more additional copies of the DNA to thereby permit overexpression or modified expression of the gene product of that DNA. In a preferred embodiment, the DNA is a cDNA copy of an mRNA transcript of a gene produced in a cell. As used herein, the terms “codon optimization” and “codon optimized” refers to a technique to maximize protein expression in fast-growing microorganisms such as Escherichia coli or Saccharomyces cerevisiae by increasing the translation efficiency of a particular gene. Codon optimization can be achieved, for example, by transforming nucleotide sequences of one species into the genetic sequence of a different species. Optimal codons help to achieve faster translation rates and high accuracy. As a result of these factors, translational selection is expected to be stronger in highly expressed genes.

As used herein, “reduced expression” refers to expression of a gene or protein at a level lower than the native expression of the gene or protein. For example, in some embodiments the activity of a reductase is reduced by decreasing the amount of protein product, or expression, of a gene encoding the reductase.

Reduction or elimination (i.e., disruption) of expression of a gene can be accomplished by any known method, including insertions, missense mutations, frame shift mutations, deletion, substitutions, or replacement of a DNA sequence, or any combinations thereof. Insertions include the insertion of the entire genes, which may be of any origin. Reduction or elimination of gene expression can, for example, comprise altering or replacing a promoter, an enhancer, or splice site of a gene, leading to inhibition of production of the normal gene product partially or completely. In some embodiments, reduction or elimination of gene expression comprises altering the total level of the protein product expressed in the cell or organism. In other embodiments, disruption of a gene comprises reducing or eliminating the activity of the protein product of the gene in a cell or organism. In some embodiments of the disclosure, the disruption is a null disruption, wherein there is no significant expression of the gene. In some embodiments the disruption of a gene in a host cell or organism occurs on both chromosomes, in which case it is a homozygous disruption. In other embodiments the disruption of a gene in a host cell or organism occurs on only one chromosome, leaving the other chromosomal copy intact, in which case it is a heterozygous gene disruption. In still other embodiments each copy of a gene in a host cell or organism is disrupted differently.

Reduction or elimination of gene expression may also comprise gene knock-out or knock-down. A “gene knock-out” refers to a cell or organism in which the expression of one or more genes is eliminated, and therefore the cell or organism does not express the one or more genes knocked-out. A “gene knock-down” refers to a cell or organism in which the level of one or more genes is reduced, but not completely eliminated.

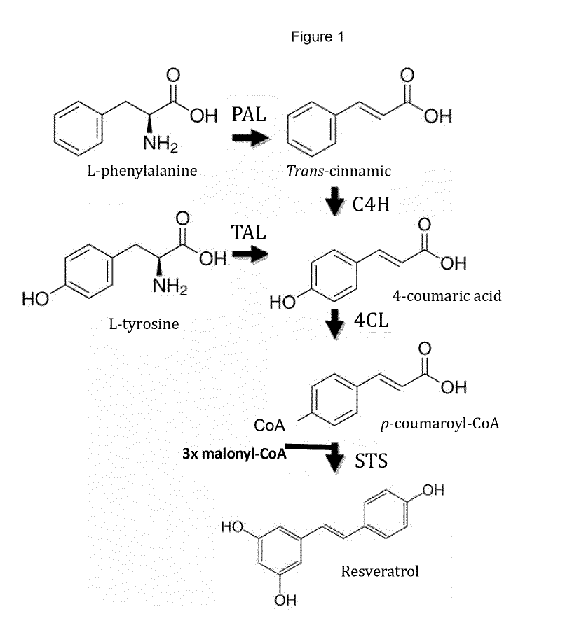

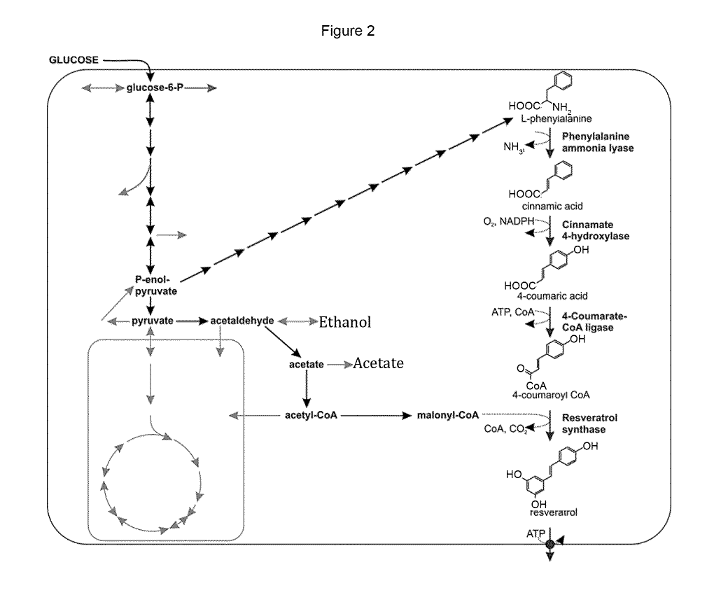

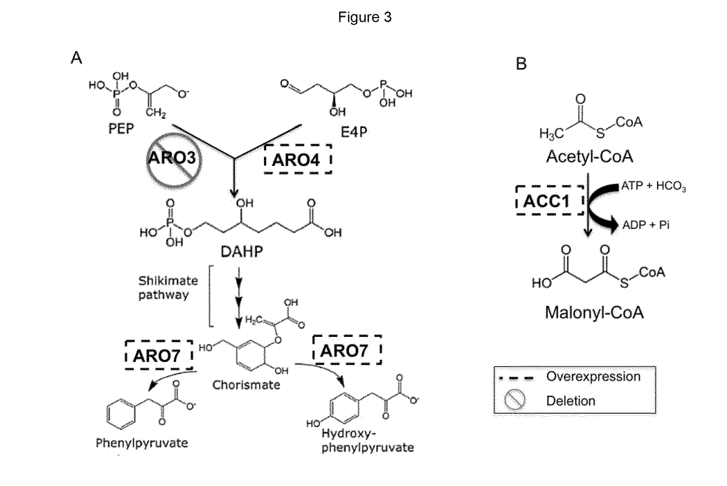

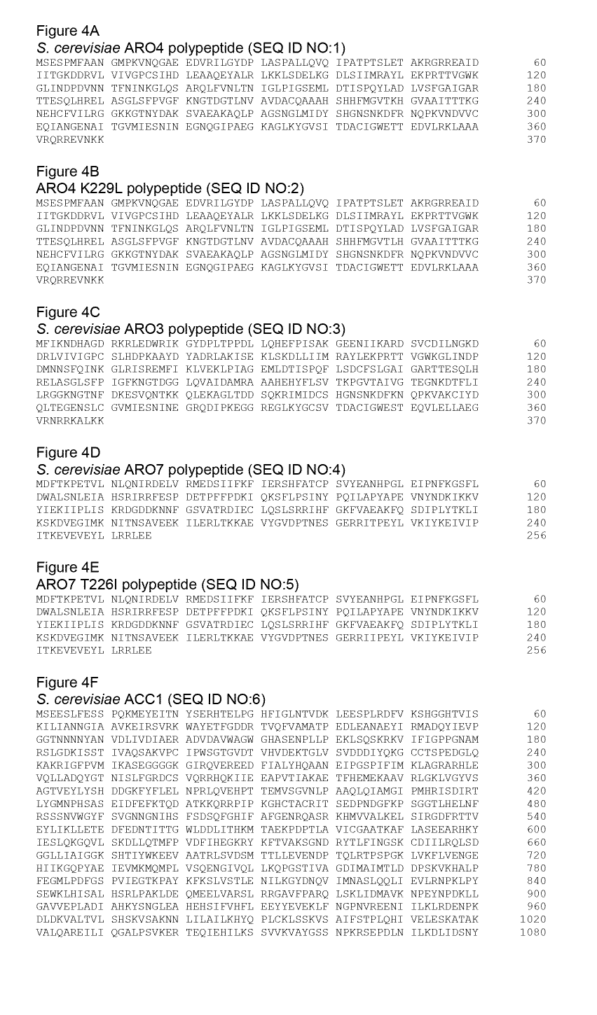

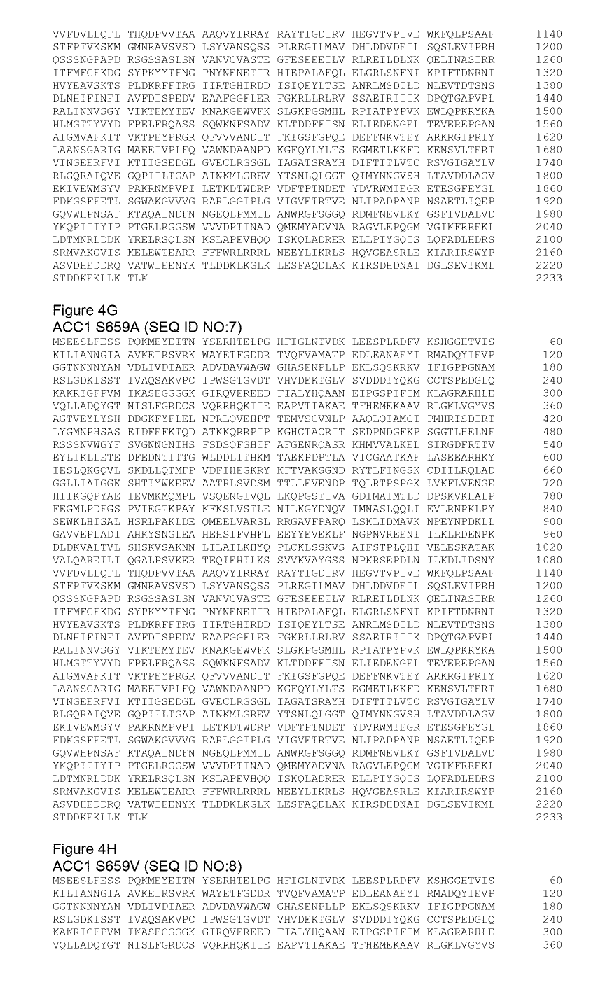

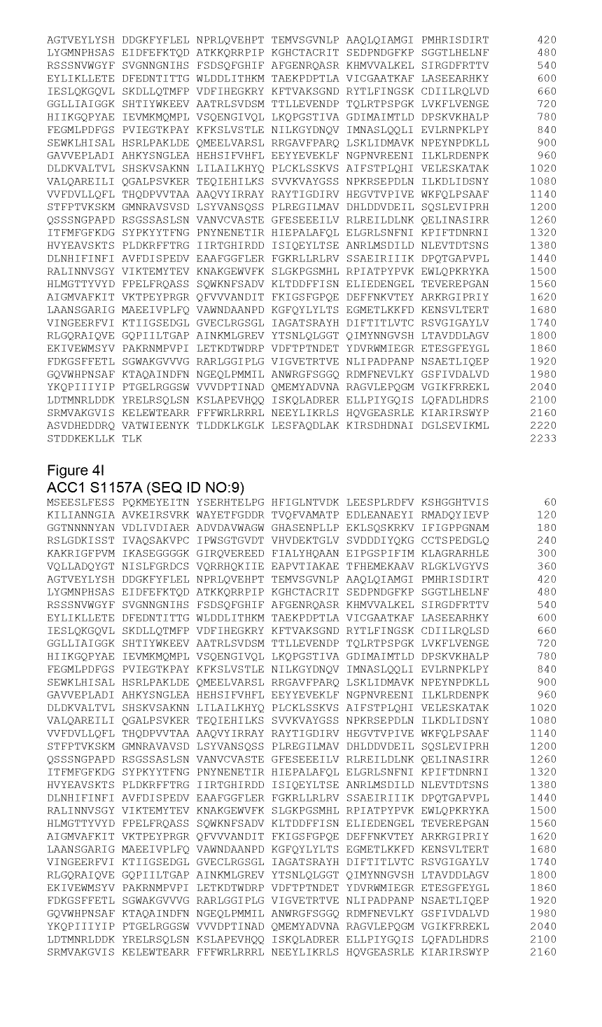

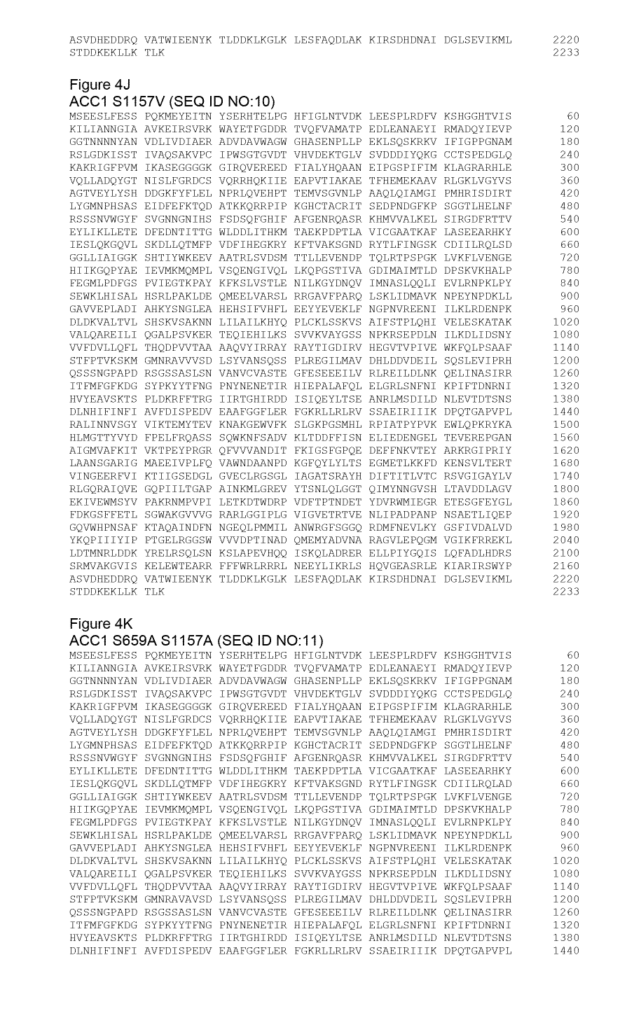

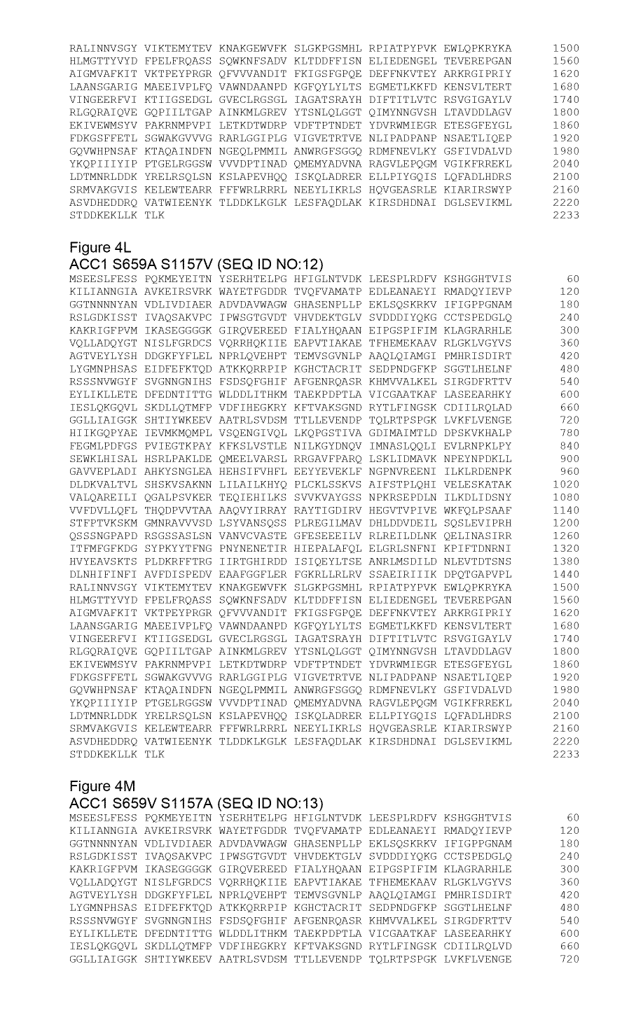

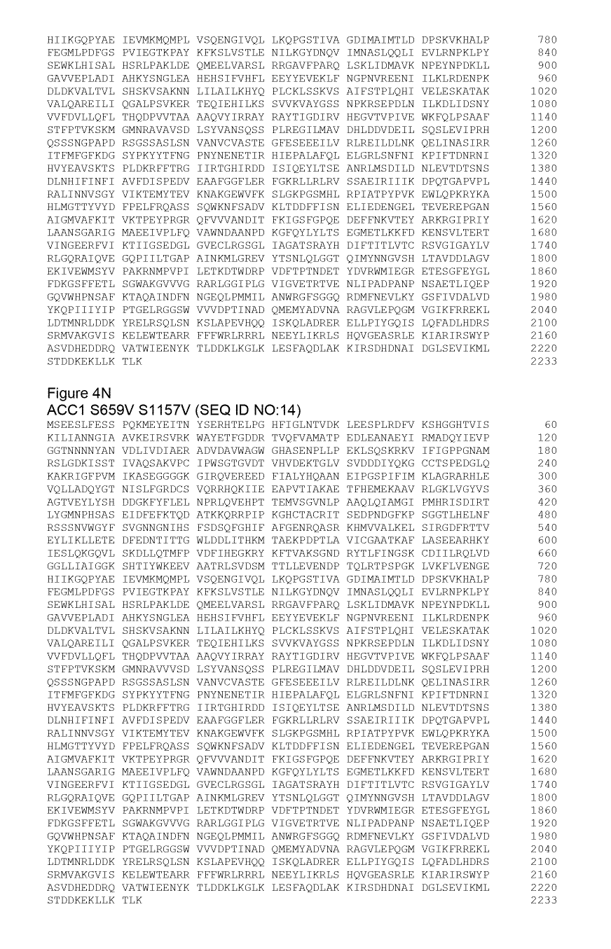

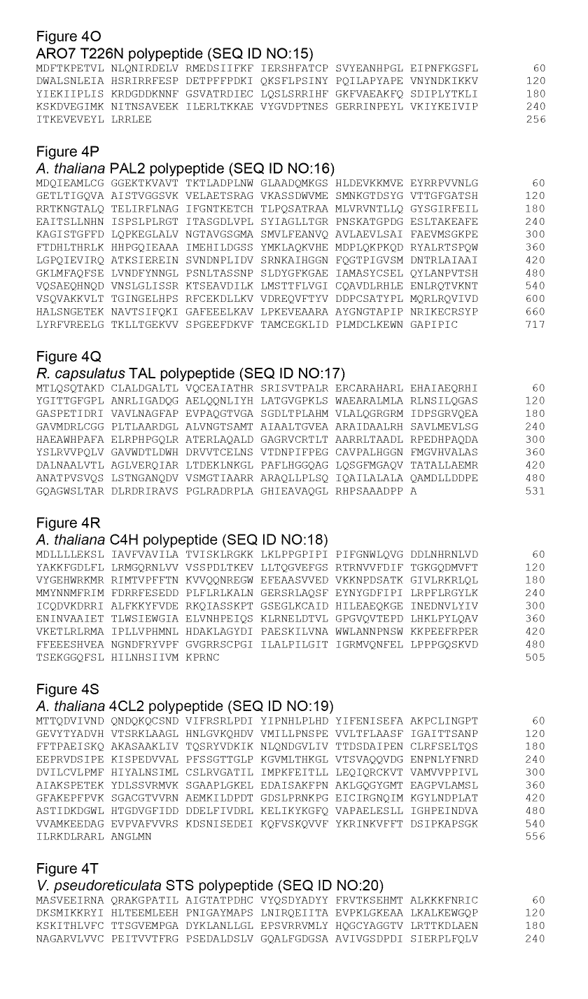

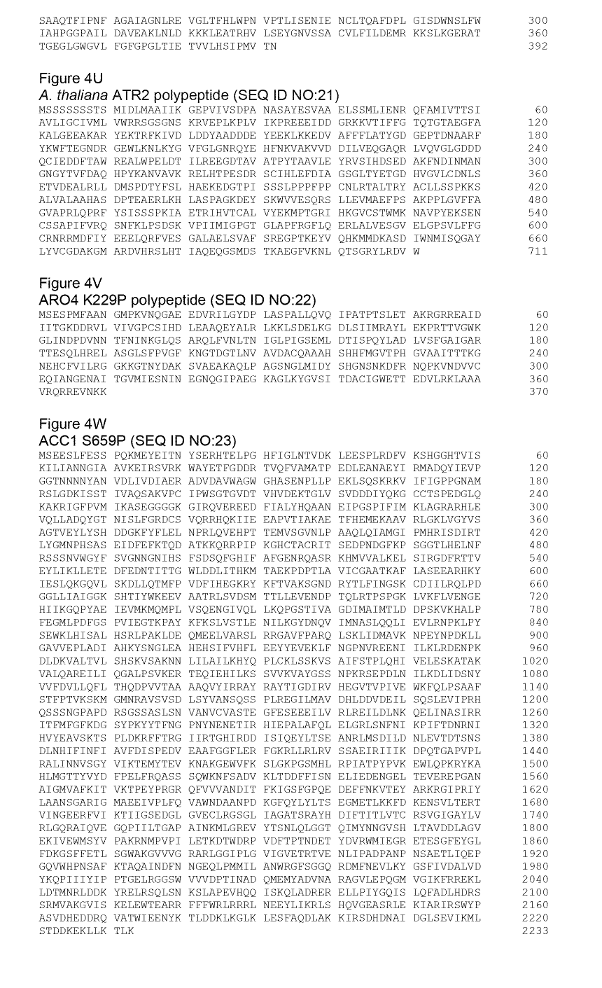

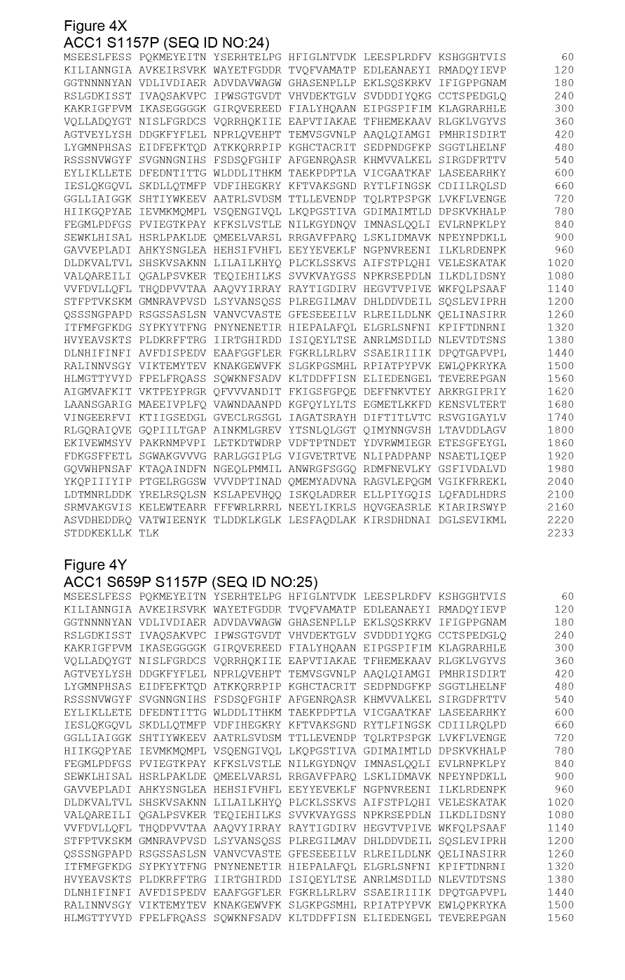

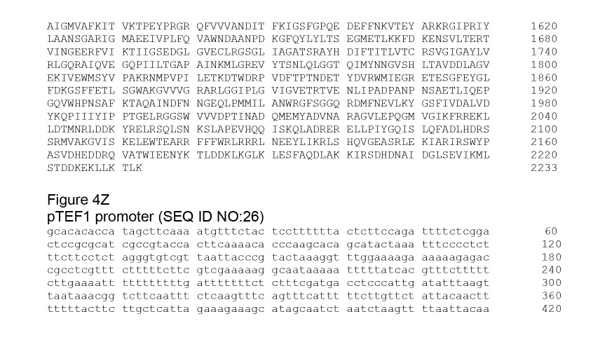

As used herein, the terms “resveratrol-producing strain,” “resveratrol-producing cells,” “resveratrol-producing host,” and “resveratrol-producing microorganism” can be used interchangeably to refer to cells that express genes encoding proteins involved in resveratrol production (see, e.g., FIGS. 1, 2). For example, a resveratrol-producing strain can express genes encoding one or more of an L-phenylalanine ammonia lyase (PAL) polypeptide, a cinnamate-4-hydroxylase (C4H) polypeptide, a cytochrome P450 monooxygenase polypeptide, an NADPH:cytochrome P450 reductase polypeptide, a 4-coumarate-CoA ligase (4CL) polypeptide, and a stilbene synthase (STS) polypeptide. In another example, a resveratrol-producing strain can express genes encoding one or more of a tyrosine ammonia lyase (TAL), a 4-coumarate-CoA ligase (4CL) polypeptide, and a stilbene synthase (STS) polypeptide. One or more of the genes encoding proteins involved in resveratrol production can be recombinant. See, e.g., WO2006/089898, WO2008/009728, WO2009/016108, WO2009/124879, WO2009/124967, WO2011/147818, which are incorporated by reference in their entirety.

In some embodiments, a stilbene-producing host comprises a gene encoding a 4-coumarate-CoA ligase (4CL) polypeptide and a gene encoding stilbene synthase (STS) polypeptide, wherein the host is capable of producing the stilbene from a carbon source when the host is fed, for example, but not limited to, coumaric acid. See, e.g., Wang et al., Annals of Microbiology, 2014, ISSN 1590-4261.

In some embodiments, a L-phenylalanine ammonia lyase (PAL) polypeptide can be expressed, overexpressed, or recombinantly expressed in said microorganism. In some embodiments, said PAL is a PAL (EC 4.3.1.5) from a plant belonging to the genus of Arabidopsis, Brassica, Citrus, Phaseolus, Pinus, Populus, Solanum, Prunus, Vitis, Zea, Agastache, Ananas, Asparagus, Bromheadia, Bambusa, Beta, Betula, Cucumis, Camellia, Capsicum, Cassia, Catharanthus, Cicer, Citrullus, Coffea, Cucurbita, Cynodon, Daucus, Dendrobium, Dianthus, Digitalis, Dioscorea, Eucalyptus, Gallus, Ginkgo, Glycine, Hordeum, Helianthus, Ipomoea, Lactuca, Lithospermum, Lotus, Lycopersicon, Medicago, Malus, Manihot, Medicago, Mesembryanthemum, Nicotiana, Olea, Oryza, Pisum, Persea, Petroselinum, Phalaenopsis, Phyllostachys, Physcomitrella, Picea, Pyrus, Quercus, Raphanus, Rehmannia, Rubus, Sorghum, Sphenostylis, Stellaria, Stylosanthes, Triticum, Trifolium, Triticum, Vaccinium, Vigna, or Zinnia or a microorganism belonging to the genus Agaricus, Aspergillus, Ustilago, Rhodobacter, or Rhodotorula. See, e.g., WO 2006/089898, which has been incorporated by reference in its entirety. In some embodiments, the PAL is an Arabidopsis thaliana PAL, e.g., A. thaliana PAL2 (SEQ ID NO:16).

In some embodiments, a tyrosine ammonia lyase (TAL) polypeptide can be expressed, overexpressed, or recombinantly expressed in said microorganism. In some embodiments, said TAL is a TAL (EC 4.3.1.5) from yeast belonging to the genus Rhodotorula or a bacterium belonging to the genus Rhodobacter. See, e.g., WO 2006/089898, which has been incorporated by reference in its entirety. In some embodiments, the TAL is a Rhodobacter capsulatus TAL, e.g., R. capsulatus TAL (SEQ ID NO:17).

In some embodiments, a cinnamate 4-hydroxylase (C4H) polypeptide can be expressed, overexpressed, or recombinantly expressed in said microorganism. In some embodiments, said C4H is a C4H (EC 1.14.13.11) from a plant belonging to the genus of Arabidopsis, Citrus, Phaseolus, Pinus, Populus, Solanum, Vitis, Zea, Ammi, Avicennia, Camellia, Camptotheca, Catharanthus, Glycine, Helianthus, Lotus, Mesembryanthemum, Physcomitrella, Ruta, Saccharum, or Vigna or from a microorganism belonging to the genus Aspergillus. See, e.g., WO 2006/089898, which has been incorporated by reference in its entirety. See, e.g., WO 2006/089898, which has been incorporated by reference in its entirety. In some embodiments, the C4H is Arabidopsis thaliana C4H (SEQ ID NO:19).

In some embodiments, a 4-coumarate-CoA ligase (4CL) polypeptide can be expressed, overexpressed, or recombinantly expressed in said microorganism. In some embodiments, said 4CL can be a 4CL (EC 6.2.1.12) from a plant belonging to the genus of Abies, Arabidopsis, Brassica, Citrus, Larix, Phaseolus, Pinus, Populus, Solanum, Vitis, Zea, e.g., Z. mays, Agastache, Amorpha, Cathaya, Cedrus, Crocus, Festuca, Glycine, Juglans, Keteleeria, Lithospermum, Lolium, Lotus, Lycopersicon, Malus, Medicago, Mesembryanthemum, Nicotiana, Nothotsuga, Oryza, Pelargonium, Petroselinum, Physcomitrella, Picea, Prunus, Pseudolarix, Pseudotsuga, Rosa, Rubus, Ryza, Saccharum, Suaeda, Thellungiella, Triticum, or Tsuga, a microorganism belonging to the genus Aspergillus, Neurospora, Yarrowia, Mycosphaerella, Mycobacterium, Neisseria, Streptomyces, or Rhodobacter, or a nematode belonging to the genus Ancylostoma, Caenorhabditis, Haemonchus, Lumbricus, Meloidogyne, Strongyloidus, or Pristionchus. See, e.g., WO 2006/089898, which has been incorporated by reference in its entirety. In some embodiments, the 4CL is an Arabidopsis thaliana 4CL, e.g., A. thaliana 4CL2 (SEQ ID NO:20).

In some embodiments, a stilbene synthase (STS) polypeptide can be expressed, overexpressed, or recombinantly expressed in said microorganism. In some embodiments, said STS is an STS (EC 2.3.1.95) from a plant belonging to the genus of Arachis, Rheum, Vitis, Pinus, Piceea, Lilium, Eucalyptus, Parthenocissus, Cissus, Calochortus, Polygonum, Gnetum, Artocarpus, Nothofagus, Phoenix, Festuca, Carex, Veratrum, Bauhinia, or Pterolobium. See, e.g., WO 2006/089898, which has been incorporated by reference in its entirety. In some embodiments, the STS is Vitus pseudoreticulata STS (SEQ ID NO:21).

In some embodiments, an NADPH:cytochrome P450 reductase (CPR) polypeptide can be expressed, overexpressed, or recombinantly expressed in said microorganism. In some embodiments, said CPR is a CPR (EC 1.6.2.4) from a plant belonging to genus Arabidopsis, e.g., A. thaliana, a plant belonging to genus Citrus, e.g., Citrus×sinensis, or Citrus×paradisi, a plant belonging to genus Phaseolus, e.g., P. vulgaris, a plant belonging to genus Pinus, e.g., P. taeda, a plant belonging to genus Populus, e.g., P. deltoides, R. tremuloides, or R. trichocarpa, a plant belonging to genus Solanum, e.g., S. tuberosum, a plant belonging to genus Vitis, e.g., Vitis vinifera, a plant belonging to genus Zea, e.g., Z. mays, or other plant genera, e.g., Ammi, Avicennia, Camellia, Camptotheca, Catharanthus, Glycine, Helianthus, Lotus, Mesembryanthemum, Physcomitrella, Ruta, Saccharum, or Vigna. See, e.g., WO 2006/089898, which has been incorporated by reference in its entirety. In some embodiments, the CPR is an Arabidopsis thaliana CPR, e.g., A. thaliana ATR2 (SEQ ID NO:22).

In some embodiments, a recombinant host capable of producing resveratrol further expresses a gene encoding a 3-Deoxy-D-arabinoheptulosonate 7-phosphate (DAHP) synthase [EC 2.7.11.27] polypeptide. As used herein, a DAHP synthase polypeptide refers to an enzyme capable of catalyzing the conversion of phosphoenolpyruvate and D-erythrose 4-phosphate to DAHP and phosphate, the first step in aromatic amino acid biosynthesis by the shikimate pathway. In some embodiments, the DAHP synthase is a S. cerevisiae DAHP synthase, e.g., S. cerevisiae ARO4 (SEQ ID NO:1).

In some embodiments, a recombinant host capable of producing resveratrol overexpresses a gene encoding a DAHP synthase polypeptide. In some embodiments, the overexpressed DAHP synthase gene encodes an ARO4 (SEQ ID NO:1) polypeptide. In some aspects, the recombinant host capable of producing resveratrol overexpresses a DAHP synthase polypeptide that is not feedback-inhibited by aromatic amino acids. For example, in some aspects, an ARO4 K229 mutant polypeptide is not feedback-inhibited by phenylalanine, tyrosine, and/or tryptophan. In some embodiments, the ARO4 mutant polypeptide is a K229L mutant (SEQ ID NO:2). In some aspects, a recombinant host overexpressing a gene encoding an ARO4 or ARO4 K229L polypeptide produces resveratrol in greater quantities than a recombinant host not overexpressing an ARO4 or an ARO4 K229L polypeptide. In some embodiments, the ARO4 mutant polypeptide is a K229P mutant (SEQ ID NO:23). In some aspects, a recombinant host overexpressing a gene encoding an ARO4 K229P polypeptide produces resveratrol in greater quantities than a recombinant host not overexpressing an ARO4 K229P polypeptide.

In some embodiments, a recombinant host capable of producing resveratrol does not express an S. cerevisiae ARO3 polypeptide (SEQ ID NO:3). In some embodiments, wherein resveratrol is produced in S. cerevisiae, the endogenous S. cerevisiae ARO3 polypeptide is deleted (knocked out). A non-limiting example of a method for knocking out ARO3 is through homologous recombination. See, e.g., Hegemann & Heick, Methods Mol Biol. 765:189-206 (2011).

In some embodiments, a recombinant host capable of producing resveratrol further expresses a gene encoding a chorismate mutase [EC 5.4.99.5] polypeptide. As used herein, a chorismate mutase polypeptide refers to an enzyme capable of catalyzing the conversion of chorismate to prephenate, a step in the synthesis of phenylalanine and tyrosine. In some embodiments, the chorismate mutase is an S. cerevisiae chorismate mutase, e.g., S. cerevisiae ARO7 (SEQ ID NO:4).

In some embodiments, a recombinant host capable of producing resveratrol overexpresses a gene encoding a chorismate mutase polypeptide. In some embodiments, the overexpressed chorismate mutase gene encodes an ARO7 (SEQ ID NO:4) polypeptide. In some embodiments, the recombinant host capable of producing resveratrol overexpresses a chorismate mutase polypeptide that is not feedback-inhibited by phenylalanine or tyrosine. For example, in some aspects, an ARO7 T226 mutant polypeptide is not feedback-inhibited. In some embodiments, the ARO7 mutant polypeptide is a T226I (SEQ ID NO:5) or T226N polypeptide (SEQ ID NO:15). In some aspects, a recombinant host overexpressing a gene encoding an ARO7, ARO7 T226I, or ARO7 T226N polypeptide produces resveratrol in greater quantities than a recombinant host not overexpressing an ARO7, ARO7 T226I, or ARO7 T226N polypeptide.

In some embodiments, a recombinant host capable of producing resveratrol further expresses a gene encoding an acetyl-CoA carboxylase [EC 2.7.11.27] polypeptide. As used herein, an acetyl-CoA carboxylase polypeptide refers to a biotin-dependent enzyme capable of catalyzing the carboxylation of acetyl-CoA to produce malonyl-CoA. In some embodiments, the chorismate mutase is an S. cerevisiae acetyl-CoA carboxylase, e.g., S. cerevisiae ACC1 (SEQ ID NO:6).

In some embodiments, a recombinant host capable of producing resveratrol overexpresses a gene encoding an acetyl-CoA carboxylase polypeptide. In some embodiments, the overexpressed chorismate mutase gene encodes an ACC1 (SEQ ID NO:6) polypeptide. In some embodiments, the recombinant host capable of producing resveratrol overexpresses an acetyl-CoA carboxylase with de-regulated activity. For example, in some aspects, one or more of the phosphorylation sites of ACC1 are mutated by replacing a phosphorylable Serine residue with a non-phosphorylable amino acid. Non-limiting examples of de-regulated ACC1 polypeptides are mutated at Ser659 and/or Ser1157. In some embodiments, ACC1 polypeptides can comprise a Ser659A or Ser659V mutation and/or a Ser1157A or Ser1157V mutation. See SEQ ID NOs:7-14. In some embodiments, ACC1 polypeptides can comprise a Ser659P mutation and/or a Ser1157P mutation. See SEQ ID NOs:23-25. In some embodiments, a recombinant host overexpressing a gene encoding a de-regulated ACC1 polypeptide produces resveratrol in greater quantities than a recombinant host not overexpressing a de-regulated ACC1 polypeptide.

Resveratrol produced according to the methods disclosed herein can be cis-resveratrol or trans-resveratrol, wherein the trans-resveratrol is a predominant species. Resveratrol and resveratrol derivatives formed and/or recovered according to the invention can be analyzed by techniques generally available to one skilled in the art, for example, but not limited to, thin layer chromatography (TLC), high-performance liquid chromatography (HPLC), ultraviolet-visible spectroscopy/spectrophotometry (UV-Vis), liquid chromatography-mass spectrometry (LC-MS), and nuclear magnetic resonance (NMR).

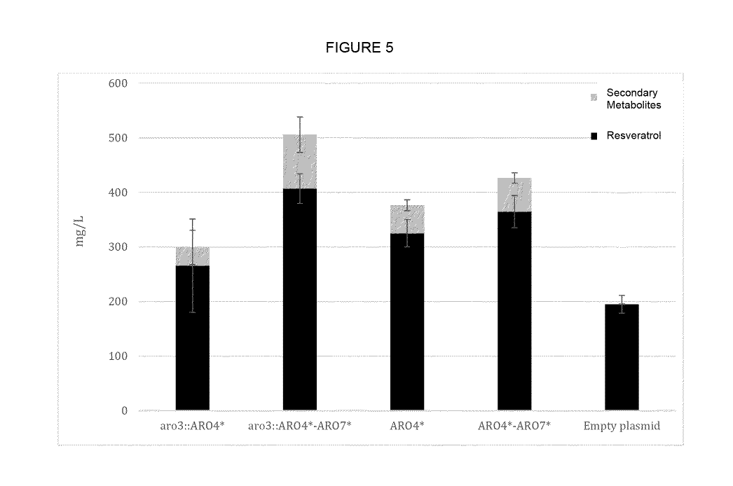

In some embodiments of the invention, resveratrol production can be increased by transforming a yeast strain with a plasmid containing a mutated version of the S. cerevisiae gene ARO4. This plasmid allows the integration of the mutated gene in a position of the yeast genome (such as, for example but not limited to, an intergenic region), which is under the control of a strong promoter (i.e. a promoter that expresses the gene above native levels, such as, for example but not limited to, pTEF1) causing overexpression of the gene. In some embodiments of the invention, the ARO4 gene contains mutations causing a change in the amino acid 229 from lysine to leucine. In some embodiments of the invention, transformants are grown for 72 hours in Delft medium at 30° C. and shaken at 400 rpm. In some embodiments of the invention, extraction of compounds of interest is performed by mixing ethanol with a culture sample to a final concentration of 50%, followed by centrifugation for 5 min at 3222×g. In some embodiments of the invention, the supernatant is analyzed by HPLC detecting, among others, resveratrol, phloretic acid and coumaric acid. As shown in FIG. 5, in some embodiments of the invention overexpression of this mutated version of ARO4 increases resveratrol production from approximately 200 mg/L in the control strain transformed with an empty plasmid to approximately 325 mg/L. Furthermore, as shown in FIG. 5, in some embodiments of the invention, overexpression of ARO4 also increases production of secondary metabolites in the pathway (i.e. the pathway intermediate coumaric acid and the side product phloretic acid are represented as secondary metabolites) as compared to the control strain transformed with empty plasmid.

In some embodiments of the invention, resveratrol production can be increased by transforming a yeast strain with a plasmid containing mutated versions of the S. cerevisiae genes ARO4 and ARO7. In some embodiments of the invention, the ARO4 gene contains mutations causing a change in the amino acid 229 from lysine to leucine. In some embodiments of the invention, the ARO7 gene contains mutations causing a change in the amino acid 226 from threonine to isoleucine. In some embodiments of the invention, the mutated ARO4 and ARO7 genes on the plasmid are inserted in a position of the yeast genome (such as, for example but not limited to, an intergenic region) and are under the control of strong promoters (i.e. a promoter that expresses the gene above native levels, such as, for example but not limited to, pTEF1 or pPGK1) causing overexpression of the genes. In some embodiments of the invention, transformants are grown for 72 hours in Delft medium at 30° C. and shaken at 400 rpm. In some embodiments of the invention, extraction of compounds of interest is performed by mixing ethanol with a culture sample to a final concentration of 50%, followed by centrifugation for 5 min at 3222×g. In some embodiments of the invention, the supernatant is analyzed by HPLC detecting, among others, resveratrol, phloretic acid and coumaric acid. As shown in FIG. 5, in some embodiments of the invention, overexpression of mutated ARO4 and ARO7 genes (ARO4*-ARO7*) increases production of resveratrol from approximately 200 mg/L in the control strain transformed with an empty plasmid to approximately 375 mg/L. In some embodiments of the invention, overexpression of mutated ARO4 and ARO7 genes also increases production of secondary metabolites in the (i.e. the pathway intermediate coumaric acid and the side product phloretic acid are represented as secondary metabolites) as compared to the control strain transformed with empty plasmid.

In some embodiments of the invention, resveratrol production can be increased by transforming a yeast strain with a plasmid containing mutated versions of the S. cerevisiae gene ARO4. In some embodiments of the invention, this plasmid allows the integration of the mutated gene at the ARO3 gene causing its disruption. In some embodiments of the invention, the mutated ARO4 gene is also under the control of a strong promoter (i.e. a promoter that expresses the gene above native levels, such as, for example but not limited to, pTEF1) causing overexpression of the gene. In some embodiments of the invention, the ARO4 gene contains mutations causing a change in the amino acid 229 from lysine to leucine. In some embodiments of the invention, the same mutated ARO4 gene is also inserted in a different position of the yeast genome not affecting the ARO3 gene, as a control to study the effect of the ARO3 deletion. In some embodiments of the invention, transformants are grown for 72 hours in Delft medium at 30° C. and shaken at 400 rpm. In some embodiments of the invention, extraction of compounds of interest is performed by mixing ethanol with a culture sample to a final concentration of 50%, followed by centrifugation for 5 min at 3222×g. As shown in FIG. 5, in some embodiments of the invention, the supernatant is analyzed by HPLC detecting, among others, resveratrol, phloretic acid and coumaric acid. In some embodiments of the invention, overexpression of the mutated version of ARO4 combined with the deletion of ARO3 (aro3::ARO4*) increases the production of resveratrol from approximately 200 mg/L in the control strain transformed with an empty plasmid to approximately 275 mg/L. Furthermore, as shown in FIG. 5, in some embodiments of the invention, the production of secondary metabolites in the pathway (i.e. the pathway intermediate coumaric acid and the side product phloretic acid are represented as secondary metabolites) are also increased for the strain overexpressing the mutated version of ARO4 combined with the ARO3 deletion (aro3::ARO4*) over the control strain transformed with an empty plasmid.

In some embodiments of the invention, resveratrol production can be increased by transforming a yeast strain with a plasmid containing mutated versions of the S. cerevisiae genes ARO4 and ARO7. In some embodiments of the invention, this plasmid allows the integration of the mutated genes at the ARO3 gene causing its disruption. In some embodiments of the invention, the mutated ARO4 and ARO7 genes are under the control of strong promoters (i.e. a promoter that expresses the gene above native levels, such as, for example but not limited to, pTEF1 or pPGK1) causing overexpression of the genes. In some embodiments of the invention, the ARO4 gene contains mutations causing a change in the amino acid 229 from lysine to leucine. In some embodiments of the invention, the ARO7 gene contains mutations causing a change in the amino acid 226 from threonine to isoleucine. In some embodiments of the invention, the same mutated ARO4 and ARO7 genes are also inserted in a different position of the yeast genome as a control to study the effect of the ARO3 deletion. In some embodiments of the invention, transformants are grown for 72 hours in Delft medium at 30° C. and shaken at 400 rpm. In some embodiments of the invention, extraction of compounds of interest is performed by mixing ethanol with a culture sample to a final concentration of 50%, followed by centrifugation for 5 min at 3222×g. In some embodiments of the invention, the supernatant is analyzed by HPLC detecting, among others, resveratrol, phloretic acid and coumaric acid. As shown in FIG. 5, in some embodiments of the invention, overexpression of the mutated versions of ARO4 and ARO7 combined with the deletion of ARO3 (aro3::ARO4*-ARO7*) increases production of resveratrol, doubling production from the control strain from approximately 200 mg/L to approximately 400 mg/L, and resulting in increased resveratrol production over the mutated ARO4 and ARO7 genes without the ARO3 deletion, from approximately 375 mg/L to approximately 400 mg/L. Also, as shown in FIG. 5, in some embodiments of the invention, overexpression of the mutated ARO4 and ARO7 genes combined with deletion of ARO3 (aro3::ARO4*-ARO7*) results in increased production of secondary metabolites in the pathway (i.e. the pathway intermediate coumaric acid and the side product phloretic acid are represented as secondary metabolites) over either the control strain transformed with empty plasmid or overexpression of mutated ARO4 and ARO7 genes without deletion of ARO3 (ARO4*-ARO7*).

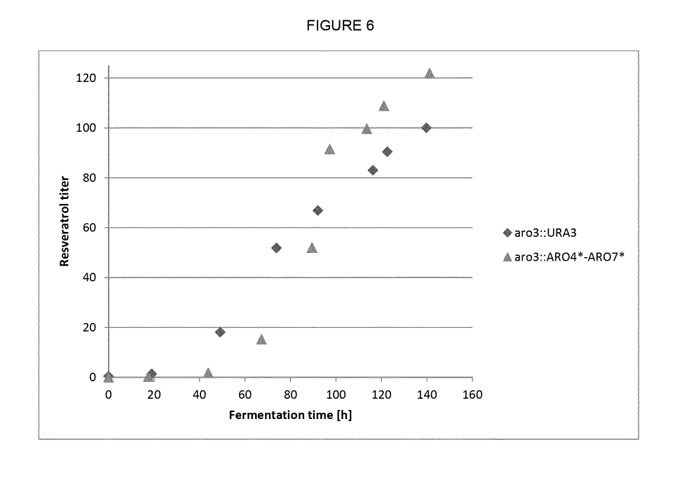

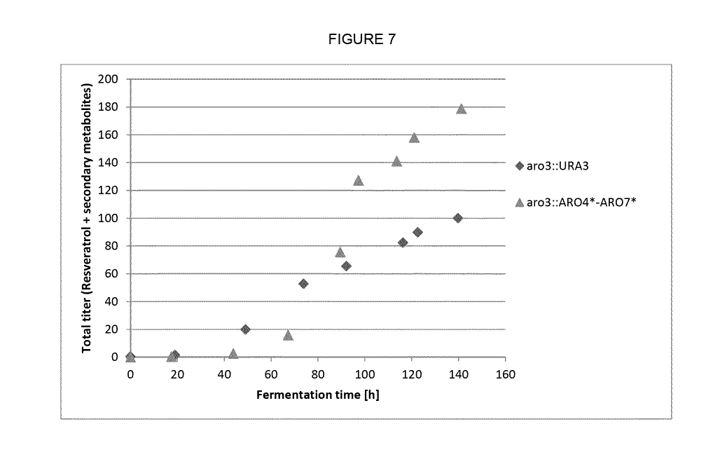

In some embodiments of the invention, resveratrol production can be increased by transforming a yeast strain with a plasmid containing mutated versions of the S. cerevisiae genes ARO4 and ARO7. In some embodiments of the invention, this plasmid allows the integration of the mutated genes at the ARO3 gene causing its disruption. In some embodiments of the invention, the mutated ARO4 and ARO7 genes are under the control of strong promoters (i.e. a promoter that expresses the gene above native levels, such as, for example but not limited to, pTEF1 or pPGK1) causing overexpression of the genes. In some embodiments of the invention, the ARO4 gene contains mutations causing a change in the amino acid 229 from lysine to leucine. In some embodiments of the invention, the ARO7 gene contains mutations causing a change in the amino acid 226 from threonine to isoleucine. In some embodiments of the invention, the resulting strain was later transformed with a DNA fragment comprising the URA3 selection marker and flanking regions, facilitating deletion of the introduced ARO4* and ARO7*. In some embodiments of the invention, cultivation of the two strains was performed in bioreactors via fed-batch culture. In some embodiments of the invention, extraction of compounds of interest was performed by mixing ethanol with a culture sample to a final concentration of 50%, followed by centrifugation for 5 min at 3222×g. In some embodiments of the invention, the supernatant was analyzed by HPLC detecting, among others, resveratrol, phloretic acid and coumaric acid. As shown in FIG. 6, in some embodiments of the invention, the results show that the strain overexpressing ARO4 and ARO7 combined with deletion of ARO3 (aro3::ARO4*-ARO7*) resulted in increased resveratrol titers by 20% over the strain with deletion of ARO3, ARO4* and ARO7* (aro3::URA3). Furthermore, as shown in FIG. 7, in some embodiments, the strain overexpressing the mutated ARO4 and ARO7 genes combined with deletion of ARO3 (aro3::ARO4*-ARO7*) resulted in approximately a 100% increase in total titer of resveratrol and secondary metabolites in the pathway (i.e. pathway intermediate coumaric acid and the side product phloretic acid are represented as secondary metabolites) over the strain with deletion of ARO3, ARO4* and ARO7* (aro3::URA3).

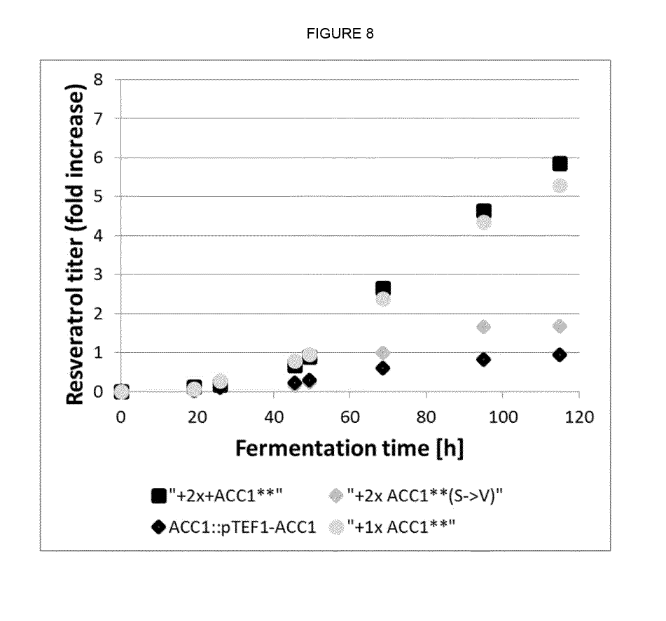

In some embodiments of the invention, resveratrol production can be increased by transforming a yeast strain with a plasmid containing a mutated ACC1 or an additional resveratrol synthase under the control of a constitutive promoter (pTEF1; SEQ ID NO:26). In some embodiments of the invention, the mutated ACC1 carried mutations conferring the residue changes S659A and S1157A; this mutant (SEQ ID NO:11) will be referred to as ACC1**. In some aspects of the invention, another resveratrol producing yeast strain, a mutated ACC1 was introduced in the same locus carrying mutations conferring the residue changes S659V and S1157V, this mutant (SEQ ID NO:14) will be referred to as ACC1** (S->V). In some aspects of the invention, the plasmid provided for integration of a single copy into the yeast genome, thereby only integrating the promoters, terminators, and genes included in the cassette in an intergenic region, without integration of the plasmid backbone itself. In some aspects of the invention, another round of integrations was carried out to introduce a second copy of the genes mentioned above. As a control to the mutated ACC1, the endogenous ACC1 gene was overexpressed by promoter replacement in a resveratrol-producing yeast strain without any additional integrated copies of mutated ACC1 (ACC1::pTEF1-ACC1). In some aspects of the invention, the promoter replacement was done by introduction of a pTEF1 promoter sequence in between the 5′ UTR of ACC1 and its start codon. In some aspects of the invention, cultivation of the transformant strains was performed in bioreactors via fed-batch culture. In some aspects of the invention, extraction of compounds was carried out by mixing ethanol with a culture sample to a final concentration of 50%, and centrifugation for 5 minutes at 3222×g. As shown in FIG. 8, the results obtained show: 1) that introduction of a mutated ACC1** resulted in elevated levels of Resveratrol final titer; 2) that both ACC1 mutant versions (ACC1** and ACC1** (S->V)) resulted in an increased Resveratrol final titer; and 3) that overexpression of a wild-type ACC1 alone did not demonstrate this improvement.

### Resveratrol Modifications

In some embodiments, a stilbene or a modified stilbene is produced by bioconversion or in a cell. In some embodiments, the stilbene is resveratrol or a resveratrol derivative. As used herein, the terms “modified resveratrol,” “resveratrol derivative,” and “resveratrol analog” can be used interchangeably to refer to a compound that can be derived from resveratrol or a compound with a similar structure to resveratrol. As used herein, the terms “resveratrol derivative” or “resveratrol analog” can be used interchangeably to refer to resveratrol-like molecules. In some embodiments, resveratrol derivatives are salts and esters of resveratrol or analogs or derivatives thereof.

Additional non-limiting examples of resveratrol analogs or derivatives thereof include hydroxylated resveratrol analogs or derivatives such as hydroxystilbene, dihydroxystilbene, 3,5-dihydroxypterostilbene, tetrahydroxystilbene, pentahydroxystilbene, or hexahydroxystilbene, fluorinated stilbenes, bridged stilbenes, digalloylresveratrol (ester of gallic acid and resveratrol), or resveratrol triacetate.

Advantageously, the resveratrol preparations of the invention have a purity defined herein as a lack or absence of chemical, biochemical or biologic contaminants present in resveratrol preparations prepared from natural sources. In exemplary embodiments, resveratrol preparations provided by the invention do not contain emodin, a plant contaminant present in resveratrol extracted from knotweed having laxative properties not desired for many applications of resveratrol.

A composition containing resveratrol or an analog or derivative thereof can be formulated into a composition and administered to a subject by any suitable route of administration, including oral or parenteral routes of administration. Specific administration modalities include subcutaneous, intravenous, intramuscular, intraperitoneal, transdermal, intrathecal, oral, rectal, buccal, topical, nasal, ophthalmic, intra articular, intra-arterial, sub arachnoid, bronchial, lymphatic, vaginal, and intra uterine administration. In some embodiments, the composition can be in the form of a capsule, liquid (e.g., a beverage), tablet, pill, gel, pellet, foodstuff, dry or wet animal feed, or formulated for prolonged release. In some embodiments, a resveratrol composition can be a solution. Any of the compositions described herein can be included in a container, pack, or dispenser together with instructions for administration. In some embodiments, the composition is packaged as a single use vial.

### Functional Homologs

Functional homologs of the polypeptides described above are also suitable for use in producing resveratrol derivatives. A functional homolog is a polypeptide that has sequence similarity to a reference polypeptide, and that carries out one or more of the biochemical or physiological function(s) of the reference polypeptide. A functional homolog and the reference polypeptide can be natural occurring polypeptides, and the sequence similarity can be due to convergent or divergent evolutionary events. As such, functional homologs are sometimes designated in the literature as homologs, or orthologs, or paralogs. Variants of a naturally occurring functional homolog, such as polypeptides encoded by mutants of a wild type coding sequence, can themselves be functional homologs. Functional homologs can also be created via site-directed mutagenesis of the coding sequence for a polypeptide, or by combining domains from the coding sequences for different naturally-occurring polypeptides (“domain swapping”). Techniques for modifying genes encoding functional polypeptides described herein are known and include, inter alia, directed evolution techniques, site-directed mutagenesis techniques and random mutagenesis techniques, and can be useful to increase specific activity of a polypeptide, alter substrate specificity, alter expression levels, alter subcellular location, or modify polypeptide-polypeptide interactions in a desired manner. Such modified polypeptides are considered functional homologs. The term “functional homolog” is sometimes applied to the nucleic acid that encodes a functionally homologous polypeptide.

Functional homologs can be identified by analysis of nucleotide and polypeptide sequence alignments. For example, performing a query on a database of nucleotide or polypeptide sequences can identify homologs of polypeptides described herein. Sequence analysis can involve BLAST, Reciprocal BLAST, or PSI-BLAST analysis of nonredundant databases using the amino acid sequence of interest as the reference sequence. Amino acid sequence is, in some instances, deduced from the nucleotide sequence. Those polypeptides in the database that have greater than 40% sequence identity are candidates for further evaluation for suitability as polypeptide useful in the synthesis of resveratrol and resveratrol derivatives. Amino acid sequence similarity allows for conservative amino acid substitutions, such as substitution of one hydrophobic residue for another or substitution of one polar residue for another. When desired, manual inspection of such candidates can be carried out in order to narrow the number of candidates to be further evaluated. Manual inspection can be performed by selecting those candidates that appear to have conserved functional domains.

Conserved regions can be identified by locating a region within the primary amino acid sequence of a polypeptide described herein that is a repeated sequence, forms some secondary structure (e.g., helices and beta sheets), establishes positively or negatively charged domains, or represents a protein motif or domain. See, e.g., the Pfam web site describing consensus sequences for a variety of protein motifs and domains on the World Wide Web at sanger.ac.uk/Software/Pfam/ and pfam.janelia.org/. The information included at the Pfam database is described in Sonnhammer et al., 1998, Nucl. Acids Res., 26:320-322; Sonnhammer et al., 1997, Proteins, 28:405-420; and Bateman et al., 1999, Nucl. Acids Res., 27:260-262. Conserved regions also can be determined by aligning sequences of the same or related polypeptides from closely related species. Closely related species preferably are from the same family. In some embodiments, alignment of sequences from two different species can be adequate.

Typically, polypeptides that exhibit at least about 40% amino acid sequence identity are useful to identify conserved regions. Conserved regions of related polypeptides exhibit at least 45% amino acid sequence identity (e.g., at least 50%, at least 60%, at least 70%, at least 80%, or at least 90% amino acid sequence identity). In some embodiments, a conserved region exhibits at least 92%, 94%, 96%, 98%, or 99% amino acid sequence identity.

A percent identity for any candidate nucleic acid or polypeptide relative to a reference nucleic acid or polypeptide can be determined as follows. A reference sequence (e.g., a nucleic acid sequence or an amino acid sequence) is aligned to one or more candidate sequences using the computer program ClustalW (version 1.83, default parameters), which allows alignments of nucleic acid or polypeptide sequences to be carried out across their entire length (global alignment). See Chenna et al., 2003, Nucleic Acids Res., 31(13):3497-500. ClustalW can be run, for example, at the Baylor College of Medicine Search Launcher site on the World Wide Web (searchlauncher.bcm.tmc.edu/multi-align/multi-align.html) and at the European Bioinformatics Institute site on the World Wide Web (ebi.ac.uk/clustalw).

To determine percent identity of a candidate nucleic acid or amino acid sequence to a reference sequence, the sequences are aligned using ClustalW, the number of identical matches in the alignment is divided by the length of the reference sequence, and the result is multiplied by 100.

It will be appreciated that polypeptides described herein can include additional amino acids that are not involved in enzymatic activities carried out by the enzyme, and thus such a polypeptide can be longer than would otherwise be the case. For example, a polypeptide can include a purification tag (e.g., HIS tag or GST tag), a chloroplast transit peptide, a mitochondrial transit peptide, an amyloplast peptide, signal peptide, or a secretion tag added to the amino or carboxy terminus. In some embodiments, a polypeptide includes an amino acid sequence that functions as a reporter, e.g., a green fluorescent protein or yellow fluorescent protein.

### Recombinant Microorganisms

A number of prokaryotes and eukaryotes are suitable for use in constructing the recombinant microorganisms described herein, e.g., bacteria, yeast and fungi. A species and strain selected for use as a strain for production of resveratrol or resveratrol derivatives first analyzed to determine which production genes are endogenous to the strain and which genes are not present (e.g., resveratrol production genes). Genes for which an endogenous counterpart is not present in the strain are assembled in one or more recombinant constructs, which are then transformed into the strain in order to supply the missing function(s).

In the present context the terms “microorganism” and “microorganism host” and “recombinant host” can be used interchangeably to refer to microscopic organisms, including bacteria or microscopic fungi, including yeast. Specifically, the microorganism can be a eukaryotic cell or immortalized cell.

Exemplary prokaryotic and eukaryotic species are described in more detail below. However, it will be appreciated that other species can be suitable. For example, suitable species can be in a genus including Agaricus, Aspergillus, Bacillus, Candida, Corynebacterium, Escherichia, Fusarium/Gibberella, Kluyveromyces, Laetiporus, Lentinus, Phaffia, Phanerochaete, Pichia, Physcomitrella, Rhodoturula, Saccharomyces, Schizosaccharomyces, Sphaceloma, Xanthophyllomyces and Yarrowia. Exemplary species from such genera include Lentinus tigrinus, Laetiporus sulphureus, Phanerochaete chrysosporium, Pichia pastoris, Physcomitrella patens, Rhodoturula glutinis 32, Rhodoturula mucilaginosa, Phaffia rhodozyma UBV-AX, Xanthophyllomyces dendrorhous, Fusarium fujikuroi/Gibberella fujikuroi, Candida utilis and Yarrowia lipolytica. In some embodiments, a microorganism can be an Ascomycete such as Gibberella fujikuroi, Kluyveromyces lactis, Schizosaccharomyces pombe, Aspergillus niger, or Saccharomyces cerevisiae. In some embodiments, a microorganism can be a prokaryote such as Escherichia coli, Rhodobacter sphaeroides, or Rhodobacter capsulatus. It will be appreciated that certain microorganisms can be used to screen and test genes of interest in a high throughput manner, while other microorganisms with desired productivity or growth characteristics can be used for large-scale production of resveratrol or resveratrol derivatives or analogs.

In certain embodiments of this invention, microorganisms include, but are not limited to, S. cerevisiae, A. niger, A. oryzae, E. coli, L. lactis and B. subtilis. The constructed and genetically engineered microorganisms provided by the invention can be cultivated using conventional fermentation processes, including, inter alia, chemostat, batch, fed-batch cultivations, continuous perfusion fermentation, and continuous perfusion cell culture.

Exemplary embodiments comprising bacterial cells include, but are not limited to, cells of species, belonging to the genus Bacillus, the genus Escherichia, the genus Lactobacillus, the genus Lactobacillus, the genus Corynebaclerium, the genus Acetobacler, the genus Acinetobacler, or the genus Pseudomonas.

The microorganism can be a fungus, and more specifically, a filamentous fungus belonging to the genus of Aspergillus, e.g., A. niger, A. awamori, A. oryzae, or A. nidulans, a yeast belonging to the genus of Saccharomyces, e.g., S. cerevisiae, S. kluyveri, S. bayanus, S. exiguus, S. sevazzi, or S. uvarum, a yeast belonging to the genus Kluyveromyces, e.g., K. laclis, K. marxianus var. marxianus, or K. thermololerans, a yeast belonging to the genus Candida, e.g., C. ulilis, C. Iropicalis, C. albicans, C. lipolylica, or C. versalilis, a yeast belonging to the genus Pichia, e.g., R. slipidis, R. pasloris, or P. sorbilophila, or other yeast genera, e.g., Cryplococcus, Debaromyces, Hansenula, Pichia, Yarrowia, Zygosaccharomyces, or Schizosaccharomyces. Concerning other microorganisms a non-exhaustive list of suitable filamentous fungi is supplied: a species belonging to the genus Penicillium, Rhizopus, Fusarium, Fusidium, Gibberella, Mucor, Morlierella, and Trichoderma.

Saccharomyces cerevisiae

Saccharomyces cerevisiae is a widely used chassis organism in synthetic biology, and can be used as the recombinant microorganism platform. There are libraries of mutants, plasmids, detailed computer models of metabolism and other information available for S. cerevisiae, allowing for rational design of various modules to enhance product yield. Methods are known for making recombinant microorganisms.

The genes described herein can be expressed in yeast using any of a number of known promoters. Strains that overproduce phenylpropanoids are known and can be used as acceptor molecules in the production of resveratrol or resveratrol derivatives.

Aspergillus spp.

Aspergillus species such as A. oryzae, A. niger and A. sojae are widely used microorganisms in food production, and can also be used as the recombinant microorganism platform. Nucleotide sequences are available for genomes of A. nidulans, A. fumigatus, A. oryzae, A. clavatus, A. flavus, A. niger, and A. terreus, allowing rational design and modification of endogenous pathways to enhance flux and increase product yield. Metabolic models have been developed for Aspergillus, as well as transcriptomic studies and proteomics studies. A. niger is cultured for the industrial production of a number of food ingredients such as citric acid and gluconic acid, and thus species such as A. niger are generally suitable for the production of resveratrol and resveratrol derivatives.

Escherichia coli

Escherichia coli, another widely used platform organism in synthetic biology, can also be used as the recombinant microorganism platform. Similar to Saccharomyces, there are libraries of mutants, plasmids, detailed computer models of metabolism and other information available for E. coli, allowing for rational design of various modules to enhance product yield. Methods similar to those described above for Saccharomyces can be used to make recombinant E. coli microorganisms.

Agaricus, Gibberella, and Phanerochaete spp.

Agaricus, Gibberella, and Phanerochaete spp. can be useful because they are known to produce large amounts of gibberellin in culture. Thus, the precursors of terpenes used as acceptor molecules in the production of resveratrol or resveratrol derivatives are already produced by endogenous genes. Thus, modules containing recombinant genes for biosynthesis of terpenes can be introduced into species from such genera without the necessity of introducing other compounds or pathway genes.

Rhodobacter spp.

Rhodobacter can be used as the recombinant microorganism platform. Similar to E. coli, there are libraries of mutants available as well as suitable plasmid vectors, allowing for rational design of various modules to enhance product yield. Isoprenoid pathways have been engineered in membraneous bacterial species of Rhodobacter for increased production of carotenoid and CoQ10. See, U.S. Patent Publication Nos. 20050003474 and 20040078846. Methods similar to those described above for E. coli can be used to make recombinant Rhodobacter microorganisms.

Physcomitrella spp.

Physcomitrella mosses, when grown in suspension culture, have characteristics similar to yeast or other fungal cultures. This genera is becoming an important type of cell for production of plant secondary metabolites, which can be difficult to produce in other types of cells.

As will be apparent to one skilled in the art, the particulars of the selection process for specific resveratrol derivatives depend on the identities of the selectable markers. Selection in all cases promotes or permits proliferation of cells comprising the marker while inhibiting or preventing proliferation of cells lacking the marker. If a selectable marker is an antibiotic resistance gene, the transfected host cell population can be cultured in the presence of an antibiotic to which resistance is conferred by the selectable marker. If a selectable marker is a gene that complements an auxotrophy of the host cells, the transfected host cell population can be cultivated in the absence of the compound for which the host cells are auxotrophic.

After selection, recombinant host cells can be cloned according to any appropriate method known in the art. For example, recombinant microbial host cells can be plated on solid media under selection conditions, after which single clones can be selected for further selection, characterization, or use. This process can be repeated one or more times to enhance stability of the expression construct within the host cell. To produce resveratrol derivatives, recombinant host cells comprising one or more expression vectors can be cultured to expand cell numbers in any appropriate culturing apparatus known in the art, such as a shaken culture flask or a fermenter.

Culture media used for various recombinant host cells are well known in the art. Culture media used to culture recombinant bacterial cells will depend on the identity of the bacteria. Culture media used to culture recombinant yeast cells will depend on the identity of the yeast. Culture media generally comprise inorganic salts and compounds, amino acids, carbohydrates, vitamins and other compounds that are either necessary for the growth of the host cells or improve health or growth or both of the host cells.

As used herein, the term “fed-batch culture” or “semi-batch culture” are used interchangeably to refer to as an operational technique in biotechnological processes where one or more nutrients (substrates) are fed (supplied) to the bioreactor during cultivation and in which the product(s) remain in the bioreactor until the end of the run. In some embodiments, all the nutrients are fed into the bioreactor.

The invention will be further described in the following examples, which do not limit the scope of the invention described in the claims.

### EXAMPLES

The Examples that follow are illustrative of specific embodiments of the invention and various uses thereof. They are set forth for explanatory purposes only, and are not to be taken as limiting the invention.

### Example 1: Resveratrol Production in a Resveratrol-Producing Strain Overexpressing Mutated ARO4

A resveratrol-producing yeast strain comprising wild-type ARO3, ARO4 and ARO7 was transformed with a plasmid containing a mutated version of the S. cerevisiae gene ARO4. This plasmid allowed integration of the mutated gene into the yeast genome, under the control of a strong promoter (pTEF1) causing overexpression of the gene. The integration occurred either in an intergenic region or at the ARO3 locus, deleting that ORF at the same time. Besides the inserted gene(s), no traces of the plasmids are inserted in the genome of the host strain. This version of the ARO4 gene contained mutations causing a change in the amino acid 229 from lysine to leucine. Transformants were grown for 72 hours in Delft medium at 30° C. and shaken at 400 rpm. Extraction of compounds of interest was performed by mixing ethanol with a culture sample to a final concentration of 50%, followed by centrifugation for 5 min at 3222×g. The supernatant was analyzed by HPLC detecting, among others, resveratrol, phloretic acid and coumaric acid. The results obtained show that overexpression of the mutated version of ARO4 increased resveratrol production from approximately 200 mg/L in the control strain transformed with an empty plasmid to approximately 325 mg/L. (FIG. 5). Overexpression of the mutated ARO4 gene also increased production of secondary metabolites in the pathway (i.e. the pathway intermediate coumaric acid and the side product phloretic acid are represented as secondary metabolites) as compared to the control strain transformed with empty plasmid. (FIG. 5).

### Example 2: Resveratrol Production in a Resveratrol-Producing Strain Overexpressing Mutated ARO4 and Mutated ARO7

A resveratrol-producing yeast strain comprising wild-type ARO3, ARO4 and ARO7 was transformed with a plasmid containing mutated versions of the S. cerevisiae genes ARO4 and ARO7. This version of the ARO4 gene contained mutations causing a change in the amino acid 229 from lysine to leucine. This version of the ARO7 gene contained mutations causing a change in the amino acid 226 from threonine to isoleucine. The mutated ARO4 and ARO7 genes on the plasmid were inserted in an intergenic region of the yeast genome and were under the control of strong promoters (pTEF1 and pPGK1, respectively) causing overexpression of the genes. Transformants were grown for 72 hours in Delft medium at 30° C. and shaken at 400 rpm. Extraction of compounds of interest was performed by mixing ethanol with a culture sample to a final concentration of 50%, followed by centrifugation for 5 min at 3222×g. The supernatant was analyzed by HPLC detecting, among others, resveratrol, phloretic acid and coumaric acid. The results obtained show that overexpression of mutated ARO4 and ARO7 genes (ARO4*-ARO7*) increased the production of resveratrol from approximately 200 mg/L in the control strain transformed with an empty plasmid to approximately 375 mg/L. (FIG. 5). Overexpression of mutated ARO4 and ARO7 also increased production of secondary metabolites in the pathway (i.e. the pathway intermediate coumaric acid and the side product phloretic acid are represented as secondary metabolites) as compared to the control strain transformed with empty plasmid. (FIG. 5).

### Example 3: Resveratrol Production in a Resveratrol-Producing ARO3 Knockout Strain Overexpressing Mutated ARO4

A resveratrol-producing yeast strain comprising wild-type ARO3, ARO4 and ARO7 was transformed with a plasmid containing a mutated version of the S. cerevisiae gene ARO4. This plasmid allowed integration of the mutated gene at the ARO3 locus, causing disruption of ARO3. The mutated ARO4 gene was also under the control of a strong promoter (pTEF1) causing overexpression of the gene. This version of the ARO4 gene contained mutations causing a change in the amino acid 229 from lysine to leucine. As detailed in Example 1, the same mutated ARO4 gene was also inserted in an intergenic region of the yeast genome, which did not affect the ARO3 gene, as a control to study the effect of the ARO3 deletion. Transformants were grown for 72 hours in Delft medium at 30° C. and shaken at 400 rpm. Extraction of compounds of interest was performed by mixing ethanol with a culture sample to a final concentration of 50%, followed by centrifugation for 5 min at 3222×g. The supernatant was analyzed by HPLC detecting, among others, resveratrol, phloretic acid and coumaric acid. The results showed that overexpression of the mutated version of ARO4 combined with deletion of ARO3 (aro3::ARO4*) increased production of resveratrol from approximately 200 mg/L in the control strain transformed with an empty plasmid to approximately 275 mg/L. (FIG. 5). However, as detailed in Example 1, overexpression of the mutated version of ARO4 without the ARO3 deletion (ARO4*) resulted in production of approximately 325 mg/L of resveratrol, approximately 50 mg/L more resveratrol than the mutated ARO4 gene combined with the deletion of ARO3. (FIG. 5). Production of secondary metabolites in the pathway (i.e. pathway intermediate coumaric acid and the side product phloretic acid are represented as secondary metabolites) also increased in the strain overexpressing the mutated version of ARO4 combined with the ARO3 deletion (aro3::ARO4*) over the control strain transformed with an empty plasmid. (FIG. 5).

### Example 4: Resveratrol Production in a Resveratrol-Producing ARO3 Knockout Strain Overexpressing Mutated ARO4 and Mutated ARO7

A resveratrol-producing yeast strain comprising wild-type ARO3, ARO4 and ARO7 was transformed with a plasmid containing mutated versions of the S. cerevisiae genes ARO4 and ARO7. This plasmid allowed integration of the mutated genes at the ARO3 locus causing its disruption. The mutated ARO4 and ARO7 genes were under the control of strong promoters (pTEF1 and pPGK1, respectively) causing overexpression of the genes. This version of the ARO4 gene contained mutations causing a change in the amino acid 229 from lysine to leucine. This version of the ARO7 gene contained mutations causing a change in the amino acid 226 from threonine to isoleucine. The same mutated ARO4 and ARO7 genes were also inserted in an intergenic region of the yeast genome as a control to study the effect of the ARO3 deletion. (See Example 2). Transformants were grown for 72 hours in Delft medium at 30° C. and shaken at 400 rpm. Extraction of compounds of interest was performed by mixing ethanol with a culture sample to a final concentration of 50%, followed by centrifugation for 5 min at 3222×g. The supernatant was analyzed by HPLC detecting, among others, resveratrol, phloretic acid and coumaric acid. The results showed that overexpression of the mutated versions of ARO4 and ARO7 combined with deletion of ARO3 (aro3::ARO4*-ARO7*) increased production of resveratrol, doubling production from the control strain from approximately 200 mg/L to approximately 400 mg/L, and resulting in increased resveratrol production over the mutated ARO4 and ARO7 genes without the ARO3 deletion, from approximately 375 mg/L to approximately 400 mg/L. (FIG. 5). Also, overexpression of the mutated ARO4 and ARO7 genes combined with deletion of ARO3 (aro3::ARO4*-ARO7*) resulted in increased production of secondary metabolites in the pathway (i.e. pathway intermediate coumaric acid and the side product phloretic acid are represented as secondary metabolites) over either the control strain transformed with empty plasmid or overexpression of mutated ARO4 and ARO7 genes without deletion of ARO3 (ARO4*-ARO7*). (FIG. 5).

### Example 5: Resveratrol Production in a Resveratrol-Producing ARO3 Knockout Strain Overexpressing Mutated ARO4 and Mutated ARO7

A resveratrol-producing yeast strain comprising wild-type ARO3, ARO4 and ARO7 was transformed with a plasmid containing mutated versions of the S. cerevisiae genes ARO4 and ARO7. This plasmid allowed integration of the mutated genes at the ARO3 locus causing its disruption. The mutated ARO4 and ARO7 genes were under the control of strong promoters (pTEF1 and pPGK1, respectively) causing overexpression of the genes. This version of the ARO4 gene contained mutations causing a change in the amino acid 229 from lysine to leucine. This version of the ARO7 gene contained mutations causing a change in the amino acid 226 from threonine to isoleucine. The resulting strain was later transformed with a DNA fragment comprising the URA3 selection marker and flanking regions, facilitating deletion of the introduced ARO4* and ARO7*. Cultivation of the two strains was performed in bioreactors via fed-batch culture. Extraction of compounds of interest was performed by mixing ethanol with a culture sample to a final concentration of 50%, followed by centrifugation for 5 min at 3222×g. The supernatant was analyzed by HPLC detecting, among others, resveratrol, phloretic acid and coumaric acid. The results show that the strain overexpressing ARO4 and ARO7 combined with deletion of ARO3 (aro3::ARO4*-ARO7*) resulted in increased resveratrol titers by 20% over the strain with deletion of ARO3, ARO4* and ARO7* (aro3::URA3). (FIG. 6). In addition, the strain overexpressing the mutated ARO4 and ARO7 genes combined with deletion of ARO3 (aro3::ARO4*-ARO7*) resulted in approximately a 100% increase in total titer of resveratrol and secondary metabolites in the pathway (i.e. pathway intermediate coumaric acid and the side product phloretic acid are represented as secondary metabolites) over the strain with deletion of ARO3, ARO4* and ARO7*(aro3::URA3) (FIG. 7).

### Example 6: Resveratrol Production in a Resveratrol-Producing Strain Overexpressing a Mutated ACC1

A resveratrol-producing yeast strain was transformed with a plasmid containing a mutated ACC1 or an additional resveratrol synthase under the control of a constitutive promoter (pTEF1; SEQ ID NO:26). The mutated ACC1 carried mutations conferring the residue changes S659A and S1157A; this mutant (SEQ ID NO:11) will be referred to as ACC1**. In another resveratrol-producing yeast strain, a mutated ACC1 was introduced in the same locus carrying mutations conferring the residue changes S659V and S1157V, this mutant (SEQ ID NO:14) will be referred to as ACC1** (S->V). The plasmid provided for integration of a single copy into the yeast genome, thereby only integrating the promoters, terminators, and genes included in the cassette in an intergenic region, without integration of the plasmid backbone itself. Another round of integrations was carried out to introduce a second copy of the genes mentioned above. As a control to the mutated ACC1, the endogenous ACC1 gene was overexpressed by promoter replacement in a resveratrol-producing yeast strain without any additional integrated copies of mutated ACC1 (ACC1::pTEF1-ACC1). The promoter replacement was done by introduction of a pTEF1 promoter sequence in between the 5′ UTR of ACC1 and its start codon. Cultivation of the transformant strains was performed in bioreactors via fed-batch culture. Extraction of compounds was carried out by mixing ethanol with a culture sample to a final concentration of 50%, and centrifugation for 5 minutes at 3222×g. The results obtained show: 1) that introduction of a mutated ACC1** resulted in elevated levels of Resveratrol final titer; 2) that both ACC1 mutant versions (ACC1** and ACC1** (S->V)) resulted in an increased Resveratrol final titer; and 3) that overexpression of a wild-type ACC1 alone did not demonstrate this improvement (FIG. 8).

Having described the invention in detail and by reference to specific embodiments thereof, it will be apparent that modifications and variations are possible without departing from the scope of the invention defined in the appended claims. More specifically, although some aspects of the present invention are identified herein as particularly advantageous, it is contemplated that the present invention is not necessarily limited to these particular aspects of the invention.

1. A recombinant host comprising:
(a) a gene encoding a 3-Deoxy-D-arabinoheptulosonate 7-phosphate (DAHP) synthase polypeptide;
(b) a gene encoding a chorismate mutase polypeptide; and
(c) a gene encoding an acetyl-CoA carboxylase polypeptide;
wherein at least one of the genes is a recombinant gene,
wherein the host is capable of producing a stilbene.

2. The host of claim 1, wherein the DAHP synthase polypeptide comprises a tyrosine-sensitive 3-Deoxy-D-arabinoheptulosonate 7-phosphate synthase (ARO4) polypeptide having at least 65% identity to the amino acid sequence set forth in SEQ ID NO: 1.

3. The host of claim 2, wherein the DAHP synthase polypeptide comprises an ARO4 polypeptide having a mutation that is K229L.

4. The host of claim 1, wherein the DAHP synthase polypeptide is not feedback inhibited.

5. The host of claim 1, wherein the host further comprises disruption of a gene encoding a phenylalanine-inhibited 3-Deoxy-D-arabinoheptulosonate 7-phosphate synthase (ARO3) polypeptide, whereby the host does not express ARO3.

6. The host of claim 5, wherein the ARO3 polypeptide has at least 75% identity to the amino acid sequence set forth in SEQ ID NO: 3

7. The host of claim 1, wherein the chorismate mutase polypeptide comprises a chorismate mutase (ARO7) polypeptide having at least 50% identity to the amino acid sequence set forth in SEQ ID NO: 4.

8. The host of claim 7, wherein the chorismate mutase polypeptide comprises an ARO7 polypeptide having a mutation that is T226I or T226N.

9. The host of claim 1, wherein the chorismate mutase polypeptide is not feedback inhibited.

10. The host of claim 1, wherein the acetyl-CoA carboxylase polypeptide comprises an acetyl-CoA carboxylase alpha (ACC1) polypeptide having at least 70% identity to the amino acid sequence set forth in SEQ ID NO: 6.

11. The host of claim 10, wherein the acetyl-CoA carboxylase polypeptide comprises an ACC1 polypeptide with a mutation at S659 or S1157, wherein the replacement amino acid is non-phosphorylable.

12. The host of claim 11, wherein the acetyl-CoA carboxylase polypeptide comprises an ACC1 polypeptide having at least one mutation that is S659A, S659V, S1157A, or S1157V.

13. The host of claim 1, wherein the acetyl-CoA carboxylase polypeptide is not feedback inhibited.

14. The host of claim 1, further comprising one or more of:
(a) a gene encoding a L-phenylalanine ammonia lyase (PAL) polypeptide;
(b) a gene encoding a cinnamate-4-hydroxylase (C4H) polypeptide;
(c) a gene encoding a NADPH:cytochrome P450 reductase polypeptide;
(d) a gene encoding a tyrosine ammonia lyase (TAL) polypeptide;
(e) a gene encoding a 4-coumarate-CoA ligase (4CL) polypeptide; or
(f) a gene encoding a stilbene synthase (STS) polypeptide;
wherein at least one of the genes is a recombinant gene.

15. The host of claim 1, wherein the stilbene is resveratrol or a resveratrol derivative.

16. The host of claim 1, wherein the host comprises a microorganism that is a yeast cell, a plant cell, a mammalian cell, an insect cell, a fungal cell, or a bacterial cell.

17. The host of claim 16, wherein the bacterial cell comprises Escherichia bacteria cells, Lactobacillus bacteria cells, Lactococcus bacteria cells, Cornebacterium bacteria cells, Acetobacter bacteria cells, Acinetobacter bacteria cells, or Pseudomonas bacterial cells.

18. The host of claim 17, wherein the yeast cell is a cell from Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactis, Hansenula polymorpha, Candida boidinii, Arxula adeninivorans, Xanthophyllomyces dendrorhous, or Candida albicans species.

19. The host of claim 18, wherein the yeast cell is a Saccharomycete.

20. The host of claim 19, wherein the yeast cell is a cell from the Saccharomyces cerevisiae species.

21. A method of producing a stilbene, comprising:
(a) growing a recombinant host in a culture medium, under conditions in which the genes are expressed,
wherein the host comprises:
(i) a gene encoding a 3-Deoxy-D-arabinoheptulosonate 7-phosphate (DAHP) synthase polypeptide;
(ii) a gene encoding a chorismate mutase polypeptide; and
(iii) a gene encoding an acetyl-CoA carboxylase polypeptide;
wherein at least one of the genes is a recombinant gene,
and, optionally
(b) recovering the stilbene from the culture media.

22. The method of claim 21, wherein the DAHP synthase polypeptide comprises an ARO4 polypeptide having at least 65% identity to the amino acid sequence set forth in SEQ ID NO: 1.

23. The method of claim 22, wherein the DAHP synthase polypeptide comprises an ARO4 polypeptide having a mutation that is K229L.

24. The method of claim 21, wherein the DAHP synthase polypeptide is not feedback inhibited.

25. The method of claim 21, wherein the host further comprises disruption of a gene encoding an ARO3 polypeptide, whereby the host does not express ARO3.

26. The method of claim 25, wherein the ARO3 polypeptide has at least 75% identity to the amino acid sequence set forth in SEQ ID NO:3.

27. The method of claim 21, wherein the chorismate mutase polypeptide comprises an ARO7 polypeptide having at least 50% identity to the amino acid sequence set forth in SEQ ID NO: 4.

28. The method of claim 27, wherein the chorismate mutase polypeptide comprises an ARO7 polypeptide having a mutation that is T226I or T226N.

29. The method of claim 21, wherein the chorismate mutase polypeptide is not feedback inhibited.

30. The method of claim 21, wherein the acetyl-CoA carboxylase polypeptide comprises an ACC1 polypeptide having at least 70% identity to the amino acid sequence set forth in SEQ ID NO:6.

31. The method of claim 30, wherein the acetyl-CoA carboxylase polypeptide comprises an ACC1 polypeptide with a mutation at S659 or S1157, wherein the replacement amino acid is non-phosphorylable.

32. The method of claim 31, wherein the acetyl-CoA carboxylase polypeptide comprises an ACC1 polypeptide having at least one mutation that is S659A, S659V, S1157A, or S1157V.

33. The method of claim 21, wherein the acetyl-CoA carboxylase polypeptide is not feedback inhibited.

34. The method of claim 21, wherein the host further comprises one or more of:
(a) a gene encoding a L-phenylalanine ammonia lyase (PAL) polypeptide;
(b) a gene encoding a cinnamate-4-hydroxylase (C4H) polypeptide;
(c) a gene encoding a NADPH:cytochrome P450 reductase polypeptide;
(d) a gene encoding a tyrosine ammonia lyase (TAL) polypeptide;
(e) a gene encoding a 4-coumarate-CoA ligase (4CL) polypeptide; or
(f) a gene encoding a stilbene synthase (STS) polypeptide;
wherein at least one of said genes is a recombinant gene.

35. The method of claim 21, further comprising the step of detecting the recovered stilbene by thin layer chromatography (TLC), high-performance liquid chromatography (HPLC), ultraviolet-visible spectroscopy/spectrophotometry (UV-Vis), liquid chromatography-mass spectrometry (LC-MS), or nuclear magnetic resonance (NMR).

36. The method of claim 21, wherein the stilbene is resveratrol or a resveratrol derivative.

37. The method of claim 1, wherein the host comprises a microorganism that is a yeast cell, a plant cell, a mammalian cell, an insect cell, a fungal cell, or a bacterial cell.

38. The method of claim 37, wherein the bacterial cell comprises Escherichia bacteria cells, Lactobacillus bacteria cells, Lactococcus bacteria cells, Cornebacterium bacteria cells, Acetobacter bacteria cells, Acinetobacter bacteria cells, or Pseudomonas bacterial cells.

39. The method of claim 37, wherein the yeast cell is a cell from Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactis, Hansenula polymorpha, Candida boidinii, Arxula adeninivorans, Xanthophyllomyces dendrorhous, or Candida albicans species.

40. The method of claim 39, wherein the yeast cell is a Saccharomycete.

41. The method of claim 40, wherein the yeast cell is a cell from the Saccharomyces cerevisiae species.
